(19) United States
(12) Patent Application Publication
(10) Pub. No.: US 2026/0196338 A1
(43) Pub. Date: Jul. 9, 2026

(54) AUTODIDACTIC DENSE ANATOMICAL MODELS IN WHICH A SELF-SUPERVISED LEARNING FRAMEWORK LEARNS TO ENCODE INHERENT PART-WHOLE HIERARCHIES WITHIN MEDICAL IMAGES

(71) Applicants: Mohammad Reza Hosseinzadeh Taher, Tempe, AZ (US); Jianming Liang, Scottsdale, AZ (US)

(72) Inventors: Mohammad Reza Hosseinzadeh Taher, Tempe, AZ (US); Jianming Liang, Scottsdale, AZ (US)

(73) Assignee: Arizona Board of Regents on Behalf of Arizona State University, Scottsdale, AZ (US)

(21) Appl. No.: 19/441,721

(22) Filed: Jan. 6, 2026

Related U.S. Application Data
(60) Provisional application No. 63/742,358, filed on Jan. 6, 2025.

(51) Int. Cl. G16H 30/40; G06N 3/0895; G06N 3/096; G06V 10/77; G06V 10/82

(52) U.S. Cl. CPC G16H 30/40; G06N 3/0895; G06N 3/096; G06V 10/7715; G06V 10/82

(57) ABSTRACT

A learning framework that learns hierarchical representations in a coarse-to-fine-manner via explicitly incorporating part-whole hierarchies into its learning objectives through three key branches: localizability, composability, and decomposability. Given an anchor whole w randomly sampled from image I, the localizability branch augments and processes w and its multi-scale views, and enforces consistency between their embeddings, yielding distinct features for different anatomical structures. The composability branch decomposes w into a set of parts and enforces consistency between the embeddings of w and the aggregated embeddings of its parts, encoding part whole relations. The decomposability branch decomposes the embeddings of w to acquire the embeddings of its constituent parts and enforce consistency between the embeddings of parts and their decomposed counterparts, capturing whole-part relations.

### CLAIM OF PRIORITY

This application claims the benefit of U.S. Provisional Patent Application No. 63/742,358, filed Jan. 6, 2025, entitled “AUTODIDACTIC DENSE ANATOMICAL MODELS IN WHICH A SELF-SUPERVISED LEARNING FRAMEWORK LEARNS TO ENCODE INHERENT PART-WHOLE HIERARCHIES WITHIN MEDICAL IMAGES”, the disclosure of which is incorporated by reference herein in its entirety.

### GOVERNMENT RIGHTS AND GOVERNMENT AGENCY SUPPORT NOTICE

This invention was made with government support under R01 HL128785 awarded by the National Institutes of Health. The government has certain rights in the invention.

### COPYRIGHT NOTICE

A portion of this document contains material which is subject to copyright protection. The copyright owner has no objection to the facsimile reproduction by anyone of the document as it appears in the Patent and Trademark Office records but otherwise reserves all copyright rights whatsoever.

### TECHNICAL FIELD

Embodiments of the invention relate generally to the field of deep learning models, particular a self-supervised learning framework that perceives human anatomy progressively in a coarse-to-fine manner, aiming to learn both anatomical (local and global) contextual information and the relative hierarchical relationships among anatomical structures.

### BACKGROUND

Human anatomy exhibits natural hierarchies. For example, the lungs are divided into the right and left lung. See, for example, FIG. 1 in which human anatomy exhibits natural hierarchies. For example, lungs are divided into right and left lungs, each with lobes. The right lung has three lobes: superior, middle, and inferior; the left lung has two lobes: superior and inferior. The pulmonary arteries, veins, and airways form hierarchical trees. These anatomy hierarchies provide inspiration for a self-supervised learning (SSL) framework (FIG. 3) that explicitly learns to encapsulate the intrinsic attributes of anatomical structures-locality and compositionality-within the embedding space, crucial for anatomy understanding, yet overlooked in existing SSL methods. In FIG. 1, each lung is divided into lobes, two on the left and three on the right lung. Each lobe is further subdivided into segments, each containing pulmonary arteries, veins, and bronchi which branch in predictable, dichotomous fashion. Consequently, anatomical structures have two important properties: (1) locality: each anatomical structure is morphologically distinct from others; and (2) compositionality and decompositionality: each anatomical structure is an integrated part of a larger whole, and vice versa, each anatomical structure (i.e., whole) can be broken down into its smaller constituent parts.

Deep learning has facilitated significant advancements in learning visual representations at multiple levels. However, the multi-level feature space learned by deep models doesn't explicitly encode part-whole hierarchies with the necessary semantic information to denote hierarchical relationships between wholes and their constituent parts. To highlight the importance of explicitly representing part-whole hierarchies in neural networks, a hypothetical system (i.e., GLOM) has been proposed to emulate human perception via parsing images into part-whole hierarchies.

### BRIEF DESCRIPTION OF THE DRAWINGS

Embodiments are illustrated by way of example, and not by way of limitation, and can be more fully understood with reference to the following detailed description when considered in connection with the figures in which:

FIG. 1 illustrates how human anatomy exhibits natural hierarchies in anatomical structures.

FIGS. 2A and 2B illustrate transforming pixels in medical images into semantically meaningful embeddings, according to the disclosed embodiments.

FIG. 3 is a functional block diagram of the disclosed embodiments.

FIG. 4 illustrates Adam-v2 effectively discriminating between anatomical landmarks.

FIGS. 5A and 5B illustrate a violin plot based on random patches extracted from images from the ChestX-ray14 dataset.

FIGS. 6A and 6B showcase Adam-v2's proficiency in understanding anatomy even when not explicitly trained for interpolation and extrapolation to understand the anatomical layouts.

FIG. 7 illustrates that Eve-v2 possesses rich dense anatomical features, co-segmenting common anatomical structures across different patients in a zero-shot setting.

FIGS. 8A and 8B illustrate that Eve-v2 recognizes anatomical symmetries, with mirrored anatomical structures associated with mirrored embeddings.

FIGS. 9A and 9B illustrate that Eve-v2 balances anatomical harmony and diversity, achieving consistent embeddings for similar anatomical structures across different scales while encoding variations in intensity, shape, boundary, and texture among instances of the same structure across patients.

FIG. 10 illustrates Adam-v2 identifying similar anatomical landmarks across multiple settings, according to the disclosed embodiments.

FIGS. 11A and 111B illustrate how Adam-v2 learns a semantically structured embedding space, where similar anatomical structures are clustered together and distinguished from dissimilar anatomical structures.

FIG. 12 graphically depicts the results of an ablation study on the impact of learning objectives, according to the disclosed embodiments.

FIG. 13 graphically depicts the results of an ablation study on the impact of coarse-to-fine learning, according to the disclosed embodiments.

FIGS. 14A and 14B show the visualization of Grad-CAM heatmaps generated according to the disclosed embodiments and baseline methods for eight diseases in ChestX-ray14.

FIG. 15 presents Table I which shows Adam-v2 preserving localizability of anatomical structures.

FIG. 16 presents Table II which demonstrates that Adam-v2 excels in few-shot transfer, outperforming fully or self-supervised learning baselines across all label fractions in anatomical structure and disease segmentation tasks, according to the disclosed embodiments.

FIG. 17 presents Table III which demonstrates that Adam-v2 yields generalizable representations for a variety of downstream tasks, according to the disclosed embodiments.

FIG. 18 presents Table IV which documents an ablation study on localizability loss.

FIG. 19 presents Table V which shows the disclosed embodiments are more robust to gender bias compared to other models.

FIG. 20 presents Table VI which demonstrates how the disclosed embodiments perform better in few-shot and full transfer settings within the fundus modality compared to the state of the art methods.

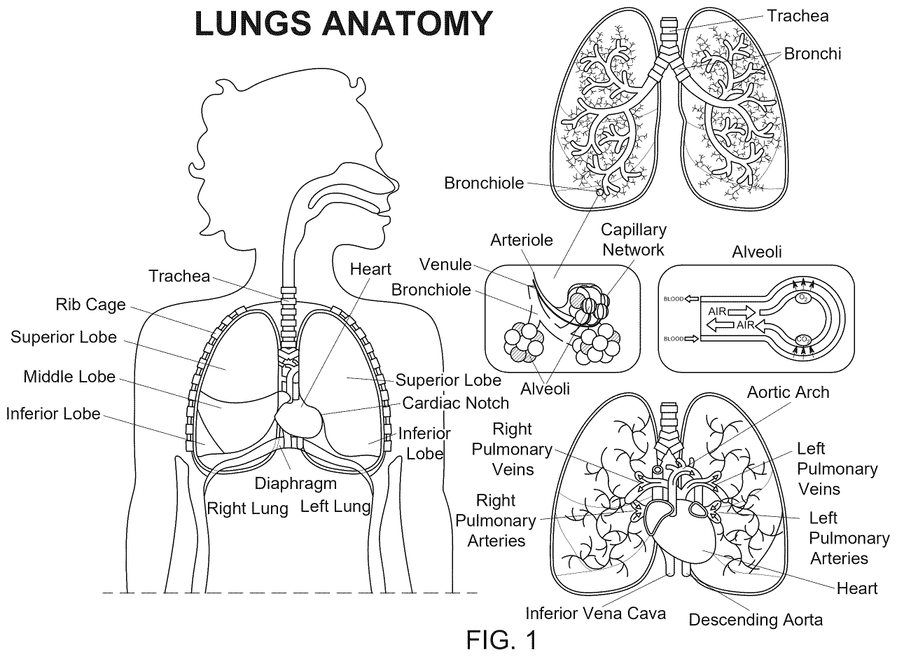

### DETAILED DESCRIPTION

### I. Introduction

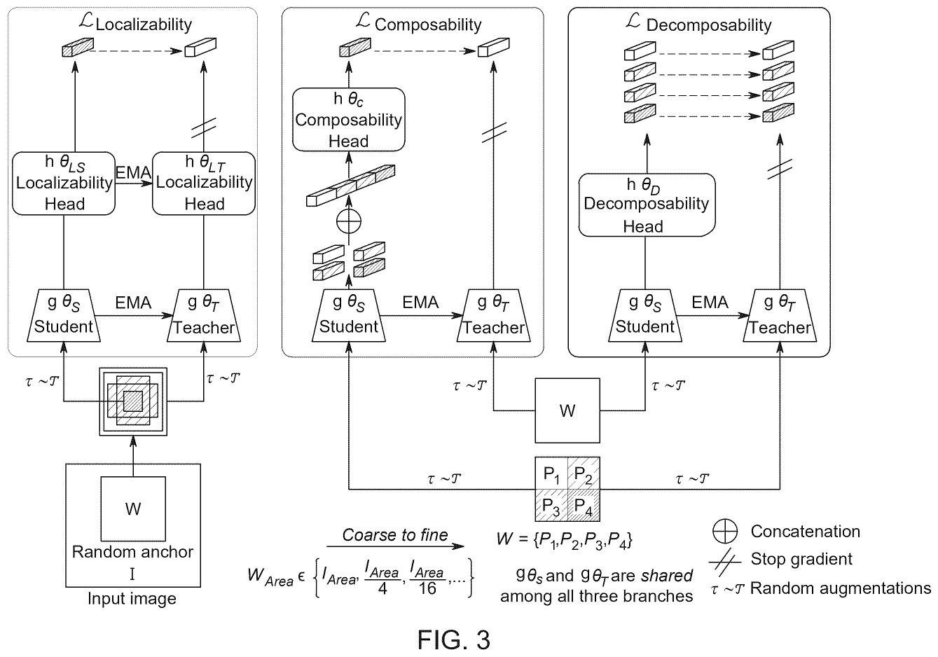

Inspired by the conceptual idea underlying GLOM, the disclosed embodiments provide a novel self-supervised learning (SSL) framework, leading to a functioning system that autodidactically constructs from medical images a hierarchy of embeddings for distinct anatomical structures, semantically balancing anatomical diversity and harmony at each level and conveying parental “whole” at a higher level and filial “parts” at a lower level. The novel framework, as illustrated in FIG. 3, comprises three branches: (1) “localizability”, which compels the model to learn a semantically structured embedding space by discriminating between different anatomical structures, (2) “composability”, which empowers the model to learn part-whole relations by constructing each anatomical structure through the integration of its constituent parts, and (3) “decomposability”, which encourages the model to learn whole-part relations by decomposing each anatomical structure into its constituent parts. Unifying these three branches together in a coarse-to-fine learning approach, the localizability branch enables the model to preserve harmony in embeddings of semantically similar anatomical structures in a hierarchy of scales. Simultaneously, composability and decomposability branches empower the model to not only convey hierarchical relationships but also preserve diversity of semantically similar anatomical structures across patients through encoding finer-grained anatomical information of their constituent parts.

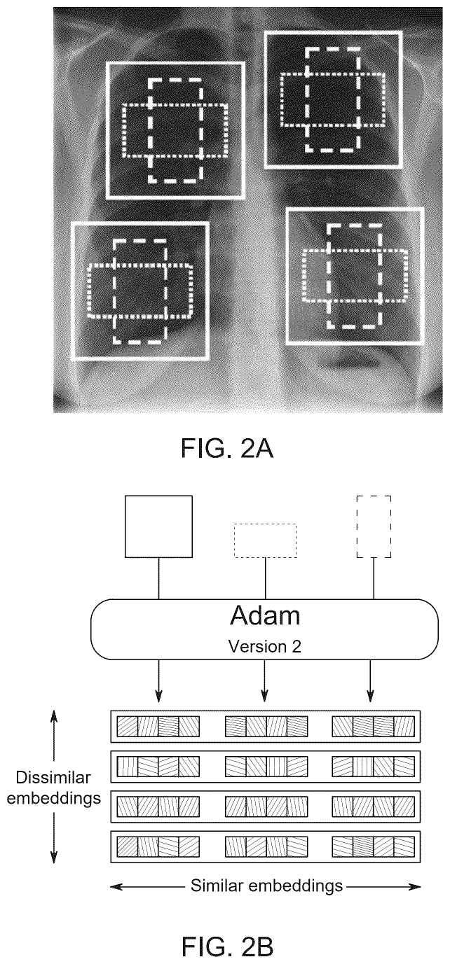

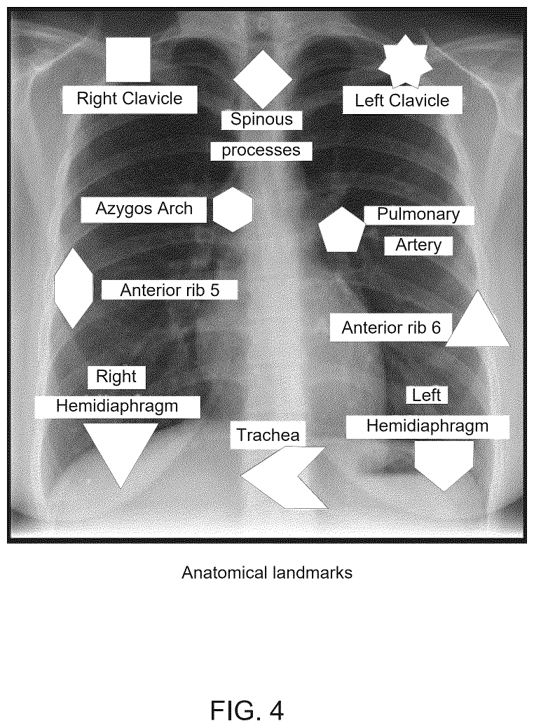

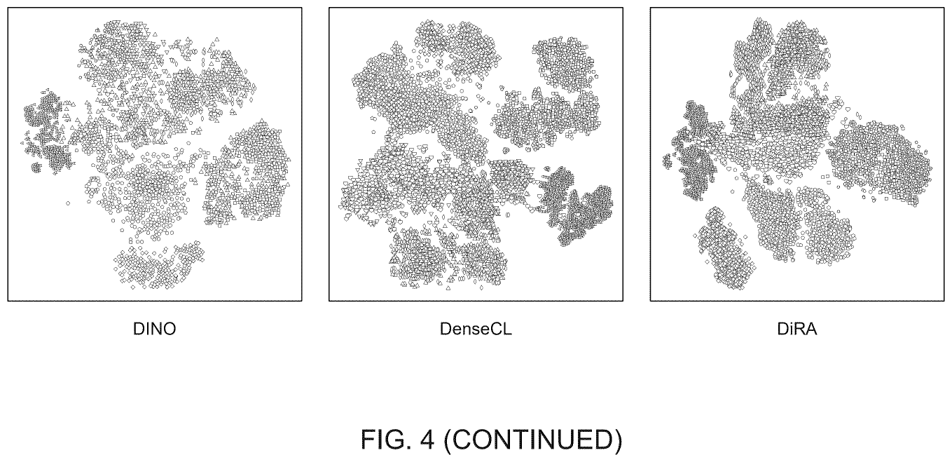

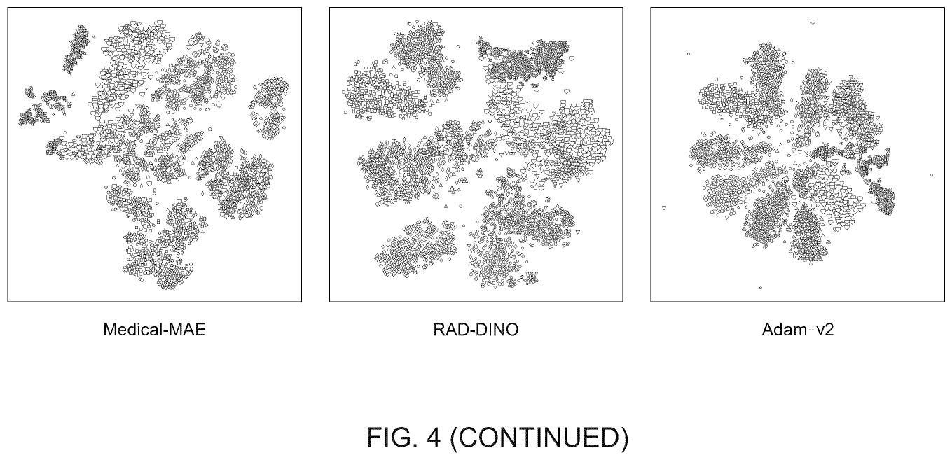

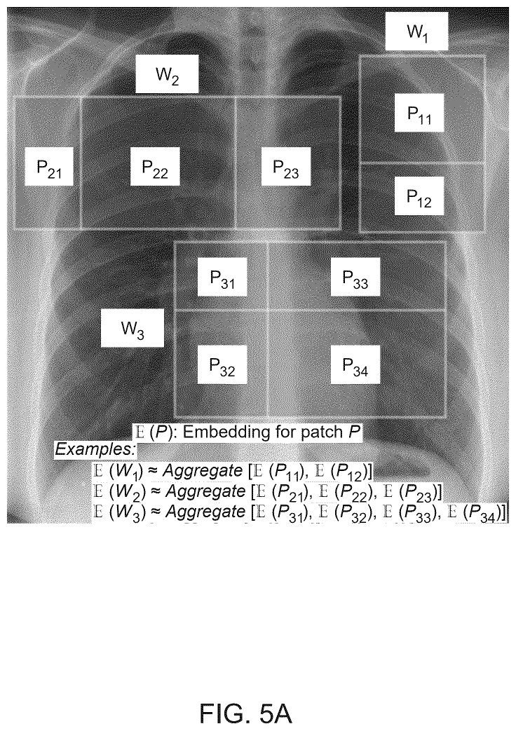

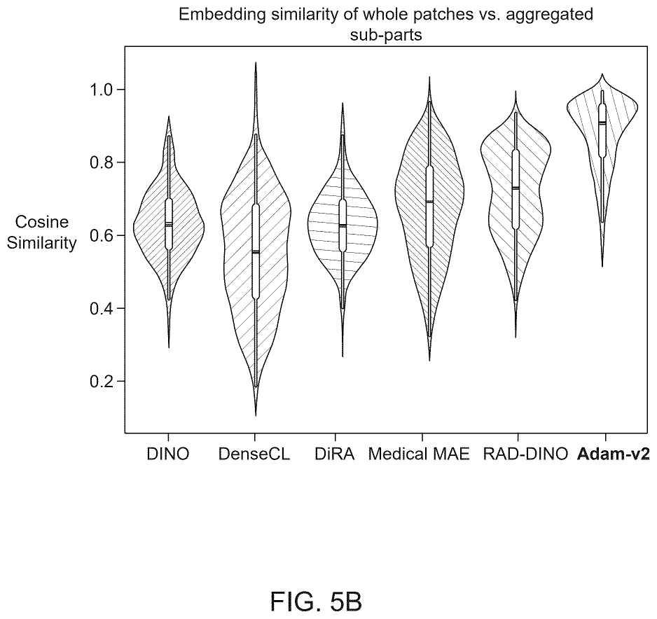

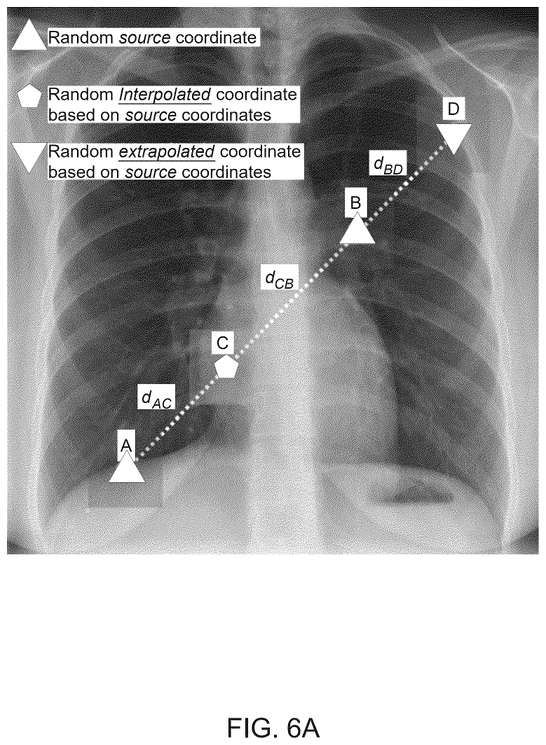

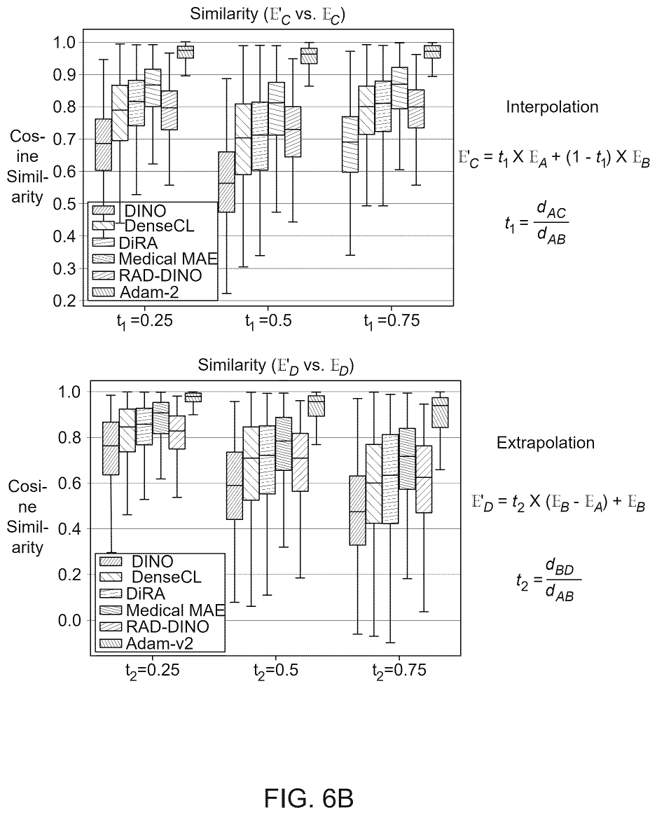

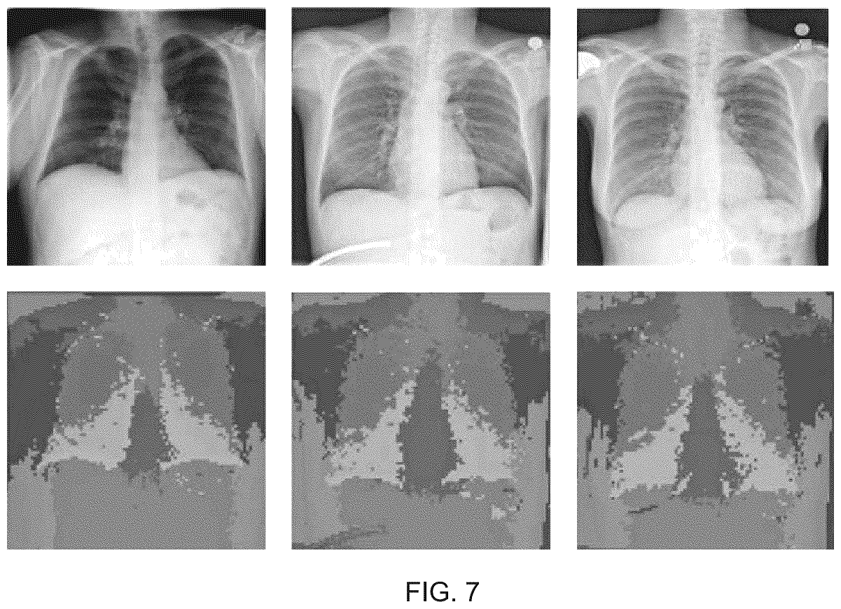

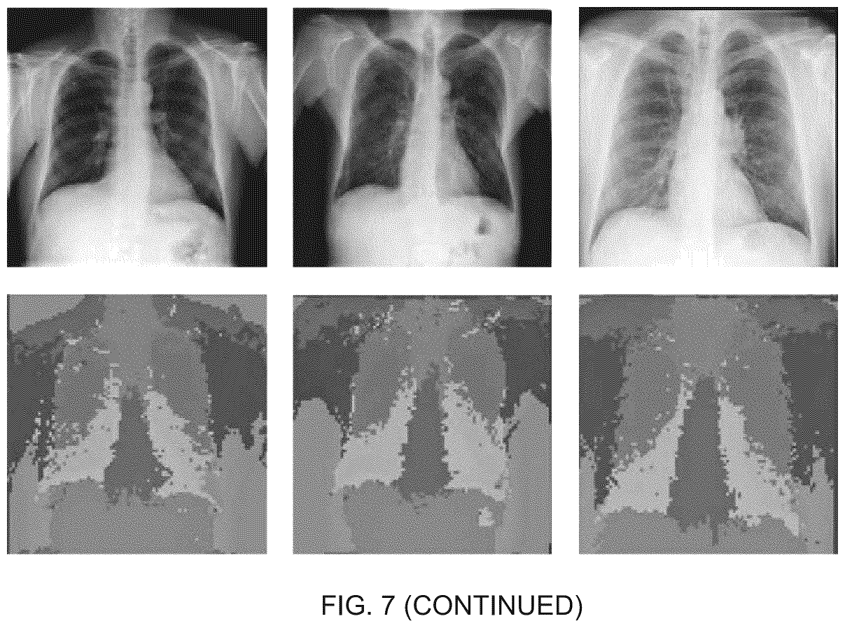

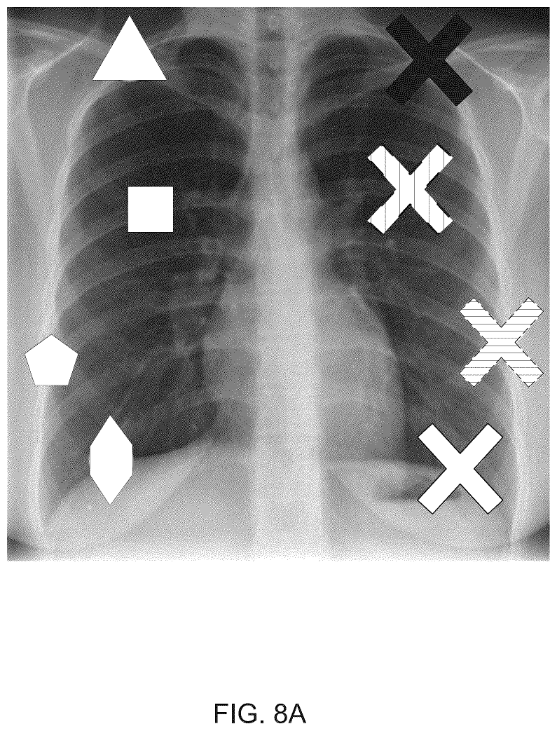

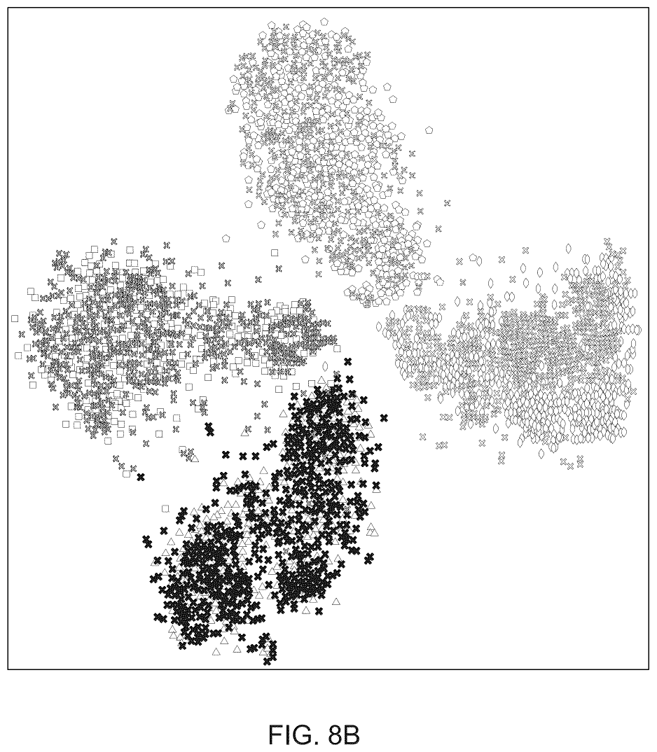

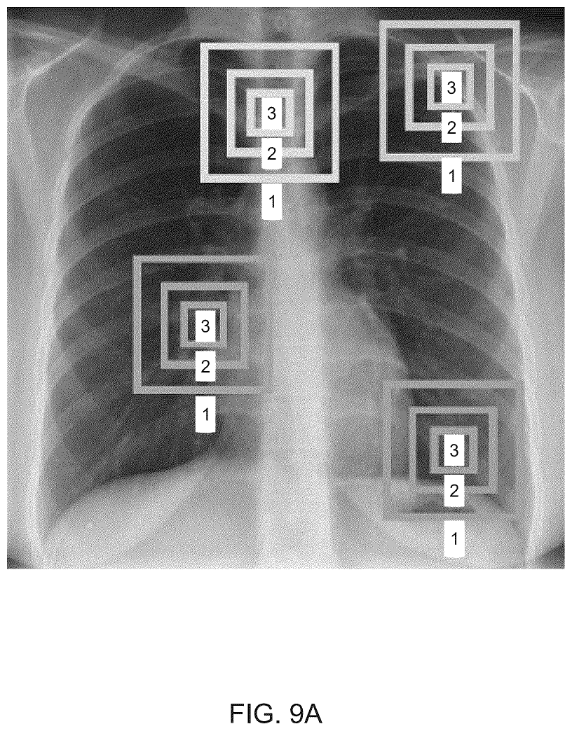

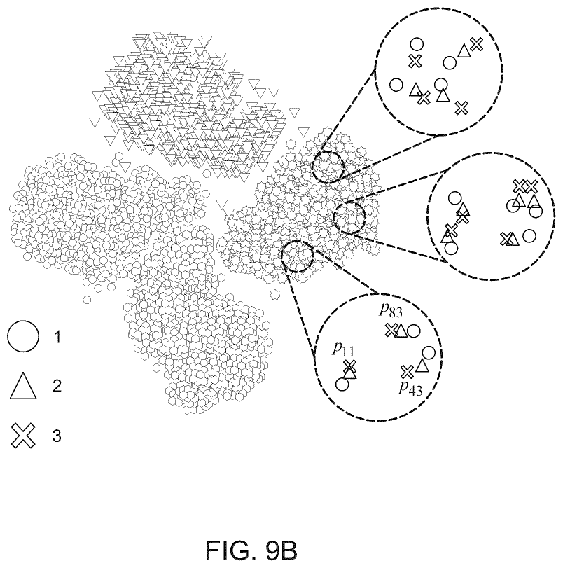

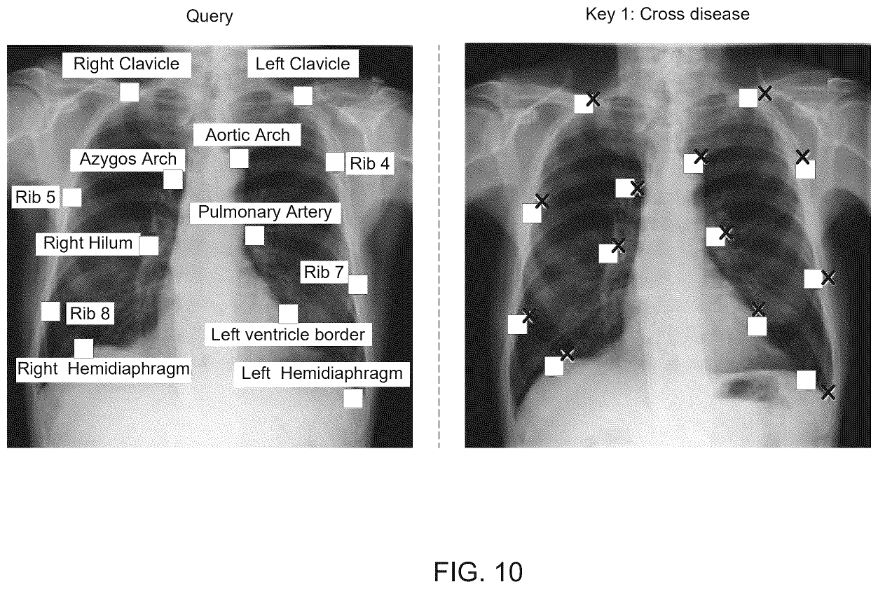

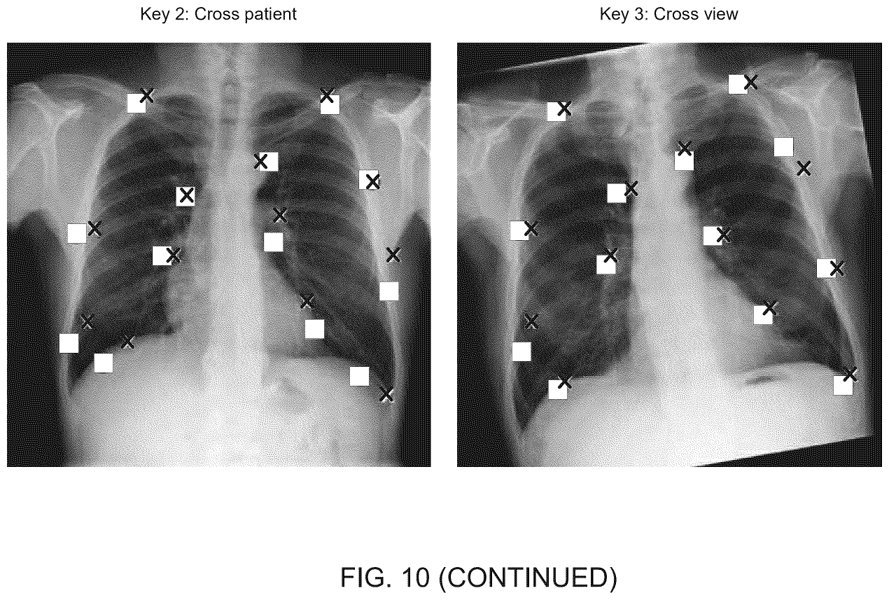

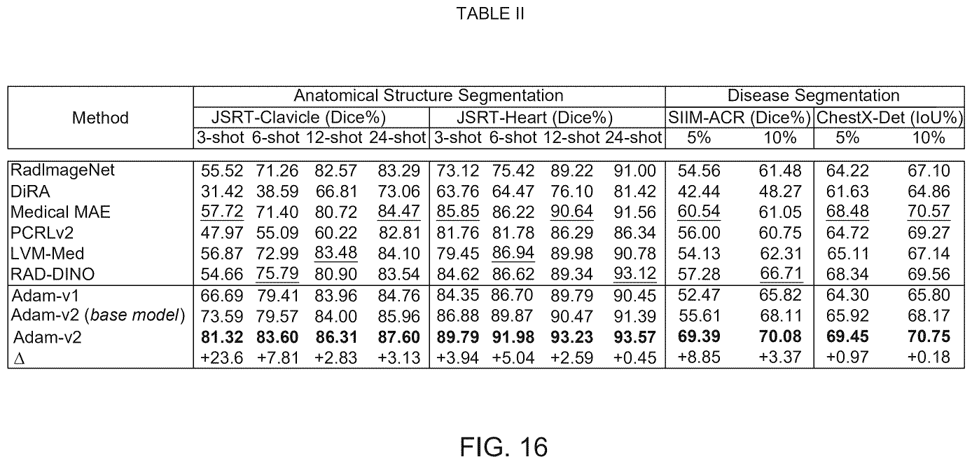

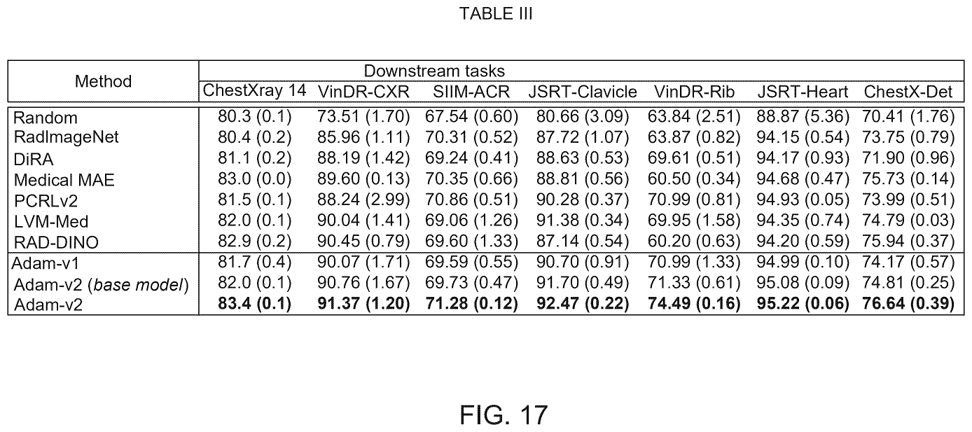

With reference to FIGS. 2A and 2B, the disclosed embodiments (i.e., a pretrained model) is referred to herein as Adam-v2 since it marks a notable improvement over a previous version—Adam (autodidactic dense anatomical models)—which learns autodidactically and produces dense anatomical embeddings, also known as Eve-v2 (embedding vectors) for its semantic richness. As illustrated in FIGS. 2A and 2B Adam-v2 transforms each pixel in medical images (e.g., chest radiographs) into semantically meaningful embeddings (Eve-v2), where different anatomical structures (indicated by different hashed boxes) are associated with different embeddings, and the same anatomical structures have (nearly) identical embeddings at all resolutions and scales (indicated by different box shapes) across patients. Eve-v2 is semantics rich as illustrated by its unique learned and emergent properties “without” any fine-tuning: (i) Eve-v2 preserves localizability of anatomical structures (FIG. 4), (ii) Eve-v2 encodes part-whole relations of anatomical structures (FIGS. 5A and 5B), (iii) Eve-v2 understands anatomical layouts via interpolation & extrapolation (FIGS. 6A and 6B), (iv) Eve-v2 associates each image pixel with (dense) semantic embeddings (FIG. 7), (v) Eve-v2 recognizes anatomical symmetries (FIGS. 8A and 8B), (vi) Eve-v2 balances anatomical harmony and diversity (FIGS. 9A and 9B), and (vii) Eve-v2 showcases robust anatomical structure matching (FIG. 10). Adam-v2 is versatile as it provides robust and generalizable representations, excelling in few-shot transfer settings (Table II presented in FIG. 16) and standing out in full transfer settings (Table III presented in FIG. 17) across various target tasks. The disclosed embodiments refer to the project site as Eden (environment for dense embeddings and networks), where all code, pretrained Adam-v2, and Eve-v2 are hosted. Comprehensive experiments reveal that (1) Eve-v2 is semantics-rich due to its unique properties crucial for understanding anatomy (See § IV-A1, § IV-A2, § IV-B1, § IV-B2, § IV-B3, § IV-B4, and § IV-B5 hereinbelow), and (2) Adam-v2 is versatile as it provides robust and generalizable representations, excelling in few-shot transfer settings (§ IV-C) and standing out in full transfer settings (§ IV-D) across various target tasks.

In summary, the disclosed embodiments provide the following contributions:
A novel self-supervised learning strategy named Adam-v2, which captures inherent hierarchical structures within medical images, producing discriminative representations infused with part-whole relationship semantics;
A new and comprehensive test suite that evaluates anatomical understanding from multiple perspectives, dissecting learned features quantitatively and qualitatively in zero-shot settings; and
A thorough set of experiments that showcase the generalizability and robustness of Adam-v2, especially highlighting its effectiveness in few-shot transfer scenarios.

### II. Method

The framework, depicted in FIG. 3, according to the disclosed embodiments, seeks to underpin the development of powerful self-supervised models foundational to medical imaging by exploiting the hierarchical nature of anatomy. Inspired by the principle of totality in Gestalt psychology-humans commonly first recognize the prominent objects in an image (e.g., lungs) and then gradually recognize smaller details based on prior knowledge about that object (e.g., each lung is divided into lobes) the disclosed embodiments provide a novel SSL strategy, which perceives the anatomy progressively in a coarse-to-fine manner, aiming to learn both anatomical (local and global) contextual information and also the relative hierarchical relationships among anatomical structures. The novel framework comprises three key branches: (1) a localizability branch which acquires discriminative representations for distinguishing different anatomical structures; (2) a composability branch which learns each anatomical structure in a parts-to-whole manner; and (3) a decomposability branch which comprehends each anatomical structure in a whole-to-parts manner. Seamlessly integrating these learning objectives into a unified framework constructs a hierarchy of embeddings from anatomy that can enhance the generalizability and robustness of representation learning. The following sections describe in detail each branch of the framework, followed by an outline of the training pipeline.

With reference to FIG. 3, the disclosed self-supervised learning framework, Adam-v2, aims to underscore the significance of constructing a hierarchy of embeddings that can be autodidactically learned from anatomy and that can enhance the generalizability and robustness of representation learning. Adam-v2 learns hierarchical representations in a coarse-to-fine-manner via explicitly incorporating part-whole hierarchies into its learning objectives through three key branches: localizability, composability, and decomposability. Given an anchor whole ω randomly sampled from image I, the localizability branch augments and processes ω and its multi-scale views, and enforces consistency between their embeddings, yielding distinct features for different anatomical structures. The composability branch decomposes ω into a set of parts and enforces consistency between the embeddings of ω and the aggregated embeddings of its parts, encoding part-whole relations. The decomposability branch decomposes the embeddings of ω to acquire the embeddings of its constituent parts and enforce consistency between the embeddings of parts and their decomposed counterparts, capturing whole-part relations. Through the seamless integration of these learning objectives into a unified framework, Adam-v2 preserves a semantic balance of anatomical diversity and harmony within its learned embedding space, yielding representations that are both generic and semantically meaningful, yet disregarded in existing SSL methods.

### 1) Localizability Branch—Acquiring Discriminative Representations to Distinguish Different Anatomical Structures.

The localizability branch aims to learn a semantically structured embedding space where similar anatomical structures are clustered together and are distinguished from dissimilar anatomical structures. As shown in FIG. 3, the localizability branch includes the student g_{Ø}_{_{S}} and teacher g_{Ø}_{_{T}} encoders, and two projectors h_{Ø}_{_{LS}} and h_{Ø}_{_{LT}}, referred to as localizability heads. The student and teacher networks share the same architecture. The parameters of student g_{Ø}_{_{S}} and localizability head h_{Ø}_{_{LS}} are optimized with stochastic gradient descent while the parameters of the teacher g_{Ø}_{_{T}} and head h_{Ø}_{_{LT}} are updated using an exponential moving average (EMA) on the weights of g_{Ø}_{_{S}} and h_{Ø}_{_{LS}}, respectively. Given an anchor patch ω randomly sampled from the input image I, a set C of multi-scale crops is extracted from ω. These crops exhibit diverse dimensions yet maintain either the same or slightly shifted center as ω, enriching the understanding of a single anatomical structure across various resolutions. Subsequently, random data augmentations T(.) are applied on ω and multiscale crops in C. The augmented view of ω is passed to the teacher, while the augmented views of the crops in C are passed to the student network, generating the features yₜ=g_{Ø}_{_{T}}(T(ω)) and γ_{S}={g_{Ø}_{_{S}}(T(c))|c∈C}, respectively. The localizability heads are employed to project the features produced by the teacher and student networks to the output embeddings z_{τ}=h_{Ø}_{_{LT}}(yₜ) and Zₛ={h_{Ø}_{_{LS}}(yₛ)|yₛ∈γₛ}, which are normalized with a softmax function:

$${{P}_{t} ( {z}_{t} )}^{( i )} = \frac{exp ( {z}_{t}^{( i )} / {τ}_{t} )}{{\sum }_{k = 1}^{K} exp ( {z}_{t}^{( i )} / {τ}_{t} )} \qquad ( 1 )$$

where τ₁>0 is a temperature parameter controlling the sharpness of the teacher's output distribution, and K is the output dimension of the localizability heads. A softmax function Pₛ with temperature τₛ is similarly employed to normalize the features in Zₛ. The localizability branch's objective is to maximize the consistency between the embedding vectors of the input anchor and its augmented views. To do so, the disclosed embodiments employ cross-entropy loss to train the localizability branch:

$${ℒ}_{Localizability} = - \frac{1}{\vert {Z}_{\text{?}} \vert} {\sum }_{{z}_{\text{?}} ∈ {Z}_{\text{?}}} {P}_{t} ( {z}_{t} ) log {P}_{\text{?}} ( {z}_{\text{?}} ) \qquad ( 2 )$$

$$\text{?} \text{indicates text missing or illegible when filed}$$

Notably, the disclosed framework provides flexibility in utilizing various localizability loss functions. While the disclosed embodiments opt for self-distillation loss due to its simplicity and efficiency, alternative objectives, like contrastive loss, may also be employed, according to the disclosed embodiments.

2) Composability Branch—Encoding the Semantics of Parts-Whole Relations from Anatomical Structures.

The composability branch aims to learn the part-whole anatomical hierarchies in a bottom-up manner by assembling larger anatomical structures from their smaller constituent subparts. As shown in FIG. 3, the composability branch consists of the student g_{Ø}_{_{S}} and teacher g_{Ø}_{_{T}} encoders, which are shared with the localizability branch, and a composability head hoc. Given an anchor whole ω randomly sampled from the input image I, the disclosed embodiments decompose it into a set of n non-overlapping parts

$$P = {\{ {p}_{i} \}}_{i = 1}^{n} .$$

The parts are augmented and processed by the student network, generating parts' embeddings

$${Y}_{ps} = {\{ {y}_{i} = {g}_{{∅}_{S}} ( T ( {p}_{i} ) ) \}}_{i = 1}^{n} .$$

The parts' embeddings are then concatenated and passed to the composability head h_{Ø}_{_{C}} to produce the aggregated embeddings of parts

$${z}_{ps} = {h}_{{∅}_{C}} ( ⊕ ( {\{ {y}_{i} \}}_{i = 1}^{n} ) ) .$$

The whole anatomical structure ω undergoes augmentation and is then passed to the teacher network to generate its corresponding embedding z_{wt}=g_{Ø}_{_{T}}(T(ω)). The composability branch is trained to maximize the agreement between the whole's embedding and the aggregated embeddings of its parts:

$${ℒ}_{Composability} = {ℓ}_{s} ( {z}_{wt} , {z}_{ps} ) \qquad ( 3 )$$

where [symbol] ₛ(z_{wt}, zₚₛ) presents a function that measures similarity between z_{wt} and zₚₛ, such as MSE, cross-entropy, or cosine similarity.
3) Decomposability Branch—Capturing the Semantics of Whole-Parts Relations from Anatomical Structures.

The decomposability branch aims to learn the whole-part anatomical hierarchies in a top-down manner by decomposing larger anatomical structures into their smaller constituent subparts. As shown in FIG. 3, the decomposability branch comprises the student g_{θ}_{_{S}} and teacher g_{θ}_{_{T}}, encoders, which are shared with the localizability and composability branches, and a decomposability head h_{θ}_{_{D}}. Given an anchor whole ω randomly sampled from the input image I, it is decomposed into a set of n non-overlapping parts

$$P = {\{ {p}_{i} \}}_{i = 1}^{n} .$$

The anchor whole ω is augmented and fed into the student network, producing the whole's embedding z_{ws}=g_{ØS}(T(ω)). The whole's embedding is then passed to the decomposability head h_{θ}_{_{D}}, which decomposes it into a set of individual embeddings corresponding to the constituent parts of the whole zₚₛ=h_{Ø}_{_{D}}(z_{ws}). Additionally, the parts

$$P = {\{ {p}_{i} \}}_{i = 1}^{n}$$

are augmented and processed by the teacher network, generating parts' embeddings

$${Z}_{pt} = {\{ {g}_{{θ}_{T}} ( 𝒯 ( {p}_{i} ) ) \}}_{i = 1}^{n} .$$

The decomposability branch is trained to maximize the agreement between embeddings of individual parts and their decomposed counterparts:

$${ℒ}_{Decomposability} = \frac{1}{\vert P \vert} {\sum }_{i = 1}^{\vert P \vert} {ℓ}_{s} ( {z}_{{p}_{i}} , {z}_{{p}_{i}^{′}} ) \qquad ( 4 )$$

where zₚ_{ᵢ}∈Zₚₜ and z_{p′}_{ᵢ}∈Zₚₛ, and [symbol] ₛ(zₚ_{ₜ},z_{p′}_{ᵢ}) presents a function that measures similarity between zₚ_{ᵢ} and z_{p′}_{ᵢ}, such as MSE, cross-entropy, or cosine similarity.

Training Pipeline. To guide the model in learning hierarchical representations, the disclosed embodiments contemplate a hierarchy of diverse anatomical structures at various scales. Specifically, the highest level of the hierarchy represents entire images (of spatial resolution (H×W)) with complete anatomy, while each subsequent level m∈{1, 2 . . . } represents anatomical structures ω at a scale of

$$\frac{H}{{2}^{m}} \times \frac{W}{{2}^{m}} ,$$

randomly sampled from the images. In a coarse-to-fine manner, the anatomical structures ω at each level are fed as the input to the localizability, composability, and decomposability branches, and are learned through the following combined loss function:

$$ℒ = {λ}_{1} * {ℒ}_{Localizability} + {λ}_{2} * {ℒ}_{Composability} + {λ}_{3} * {ℒ}_{Decomposability} \qquad ( 5 )$$

where λ₁, λ₂, λ₃ are coefficients denoting the weight of each loss term. Through a unified training scheme, a rich embedding space is learned, preserving harmony among similar anatomical structures and encoding their hierarchical relations, according to the disclosed embodiments.

In particular, the localizability loss term encourages the model to capture distinctive embeddings for different anatomical structures across varying scales. Moreover, the composability and decomposability loss terms empower the model with a profound understanding of the part-whole relations in both bottom-up and top-down manners.

### III. Experiments

### A. Pretraining Adam-v2

Unlabeled chest radiographs and color fundus photographs have been used to pretrain Adam-v2 on multiple imaging modalities. To do so, a ResNet-50 backbone was used to train two Adam-v2 base models with unlabeled images from the training sets of the ChestX-ray14 and EyePACS datasets for chest X-ray and fundus imaging tasks, respectively. Additionally, to demonstrate the scalability of the framework, a large-scale Adam-v2 model was trained with a ConvNeXt-B backbone using a large amount of 926K chest X-ray images collected from the training sets of 13 publicly-available datasets, including ChestX-ray14, CheXpert, VinDr-CXR, NIH Shenzhen CXR, RSNA Pneumonia Detection Challenge, MIMIC-CXR, PadChest, COVID-19 Radiography Database, Indiana ChestX-ray, Mendeley-v2, COVIDx, JSRT, and NIH Montgomery. The localizability heads h_{Ø}_{_{LS}} and h_{Ø}_{_{LT}} include 3-layer multi-layer perceptrons (MLP) with hidden dimension 2048 and output dimension K=65536. The composability hoc and decomposability h_{Ø}_{_{D}} heads consist of 2-layer MLP with hidden dimensions 2048. AdamW optimizer with a base learning rate of 1e-3, 10 warm-up epochs, and a cosine schedule for learning rate decay and weight decay was used. The parameters of the teacher network and the localizability head h_{Ø}_{_{LT}} are updated using an exponential moving average (EMA) of the weights from the student network and h_{Ø}_{_{LS}}, respectively. The update rules are given by θ_{T}←λθ_{T}+(1−λ)θ_{S} and θ_{LT}←λθ_{LT}+(1−λ)θ_{LS}, where λ follows a cosine schedule from 0.996 to 1 throughout the training process. Centering and sharpening for the teacher's outputs are employed to prevent collapsing solutions during localizability learning. In the localizability branch, one global crop of size 224² is extracted from the input ω, along with eight multi-scale crops of size 96². For composability and decomposability branches, input resolution 224² is used. The temperature τₛ is set to 0.1, and τₜ follows a linear warm-up as described in M. Caron, H. Touvron, I. Misra, H. J'egou, J. Mairal, P. Bojanowski, and A. Joulin, “Emerging properties in self-supervised vision transformers,” in Proceedings of the IEEE/CVF International Conference on Computer Vision (ICCV), October 2021, pp. 9650-9660. Data augmentations T(.) include random color jittering, Gaussian blur, and rotation. MSE is employed as the similarity function [symbol] ₛ(.) in Eqs. 3 and 4. λ1, λ2, λ3 are equally set to 1 and n is set to 4. Adam-v2 is trained in a coarse-to-fine manner to learn hierarchical anatomical structures, incorporating diverse anatomical features at various scales. The disclosed embodiments start with m=0, where the model is trained on the entire anatomy (i.e. whole images). The disclosed embodiments then progressively decrease the anatomical structures' scales by powers of 2. In particular, for input images with spatial resolution (H×W), anatomical structures are randomly sampled with resolutions of

$$( \frac{H}{{2}^{m}} \times \frac{W}{{2}^{m}} ) ,$$

where m∈{1, 2, . . . } and used as inputs to the model during the pretraining process. For each scale, the model is trained using the objective function in Eq. 5. Anatomical structure granularity is investigated across up to 4 scales (i.e., m∈{0, 1, 2, 3}), yet an ablation study reveals that up to three scales suffice to yield robust representations. The ResNet-50 model is trained from scratch using a batch size of 512 distributed across 8 Nvidia V100-32Gb GPUs. The disclosed embodiments first warm up the localizability branch with a scheme as (200-200-100 epochs for m=0, 1, 2), empowering the model with an initial ability to discriminate different anatomical structures. Subsequently, the composability and decomposability losses are combined into the training process, and the entire framework is jointly trained (10-90-165 epochs for m=0, 1, 2). The ConvNeXt-B model is initialized from an ImageNet-22K pretrained model and is trained using a batch size of 160 with 8 GPUs. The disclosed embodiments first warm up the localizability branch (70-70-30 epochs for m=0, 1, 2) and then train the model with all three losses (10-50-30 epochs for m=0, 1, 2).

### B. Downstream Tasks

A comprehensive assessment of Adam-v2 was conducted in various settings, including zero-shot, few-shot transfer, weakly-supervised, and full fine-tuning scenarios, across 10 downstream tasks using nine public datasets spanning chest X-ray and fundus modalities. These evaluations rigorously measure Adam-v2's efficacy across applications such as anatomy understanding, binary and multi-label classification, and organ/lesion segmentation, covering diseases like tuberculosis, pneumothorax, and lung nodules across different imaging modalities, including chest radiography and fundus photography. The following description provides details of the downstream tasks incorporated in the study.

Task 1: Clavicle segmentation. This task encompasses pixel-level segmentation of the left and right clavicles. For this task, the Japanese Society of Radiological Technology (JSRT) dataset was used, which contains 247 chest radiographs (CXRs) with clavicle segmentation masks. The dataset is split into two folds (124 and 123 images). The disclosed embodiments follow the official patient-wise split, using fold-1 for training and fold-2 for testing. Mean Dice score is used as the evaluation metric to assess clavicle segmentation performance.

Task 2: Heart segmentation. This task involves pixel-level segmentation of the heart. For this task, the disclosed embodiments use the JSRT dataset, which contains 247 CXRs with heart segmentation masks. The disclosed embodiments follow the official patient-wise split, using fold-1 (124 images) for training and fold-2 (123 images) for testing. Mean Dice score is used as the evaluation metric to assess heart segmentation performance.

Task 3: Ribs segmentation. This task entails pixel-level segmentation of 20 individual ribs. For this task, the disclosed embodiments use the VinDr-Rib dataset, which contains 245 CXRs with segmentation masks for 20 individual anterior and posterior ribs (10 per lung side). The disclosed embodiments follow the official dataset split, using 196 CXRs for training and 49 for testing. This multiclass segmentation task is evaluated using the mean Dice score to assess the performance of individual rib segmentation.

Task 4: Thoracic diseases segmentation. This task entails pixel-level segmentation of 13 thoracic diseases. For this task, the disclosed embodiments use the ChestX-Det dataset, which contains 3,578 CXRs with segmentation masks for atelectasis, calcification, cardiomegaly, consolidation, diffuse nodule, effusion, emphysema, fibrosis, fracture, mass, nodule, pleural thickening, and pneumothorax, provided by board-certified radiologists. The disclosed embodiments follow the official dataset split, using 3,025 CXRs for training and 553 for testing. Intersection over Union (IoU) is used to assess thoracic disease segmentation performance.

Task 5: Pneumothorax segmentation. This task focuses on pixel-level segmentation of pneumothorax disease. For this task, the disclosed embodiments use the SIIM-ACR dataset, which comprises 10,000 CXRs accompanied by segmentation masks for pneumothorax disease, if present in an image. For training and testing, the disclosed embodiments randomly divide the dataset into 8,000 and 2,000 CXRs, respectively. Mean Dice score is used as the evaluation metric to assess pneumothorax segmentation performance.

Task 6: Common thoracic diseases classification. This task involves multi-label classification of 5 common thoracic diseases. For this task, the disclosed embodiments use the VinDR-CXR dataset, which includes 18,000 CXRs with image-level labels provided by expert radiologists for 6 conditions (lung tumor, pneumonia, tuberculosis, other diseases, COPD, and No finding). The disclosed embodiments follow the official dataset split, using 15,000 CXRs for training and 3,000 for testing. Mean AUC score is used to evaluate thoracic disease classification performance.

Task 7: Tuberculosis classification. This task involves detection of tuberculosis disease. For this task, the disclosed embodiments use the NIH Shenzhen CXR dataset, which comprises 662 CXRs (326 normal, 336 tuberculosis). The disclosed embodiments randomly split the data into 80% training and 20% testing. Mean AUC score is used to evaluate tuberculosis disease classification performance.

Task 8: Thoracic diseases classification. This task encompasses multi-label classification of 14 thoracic diseases. For this task, the disclosed embodiments use the ChestX-ray14 dataset curated by the National Institutes of Health Clinical Center, USA, which comprises 112,120 de-identified CXRs from 30,805 unique patients, with labels indicating the absence or presence of 14 thoracic disease categories. the disclosed embodiments follow the official patient-wise split, using 86 K CXRs for training and 25 K for testing. The AUC metric is employed to evaluate the performance of classification across 14 thoracic diseases.

Task 9: Retinal vessel segmentation. This task entails pixel-level segmentation of retinal vessels. For this task, the disclosed embodiments use the DRIVE dataset, which comprises 40 color fundus images accompanied by expert annotations for retinal vessels. The disclosed embodiments follow official dataset split, using 20 images for training and 20 for testing. Mean Dice score is used as the evaluation metric to assess retinal vessels segmentation performance.

Task 10: Optic disk segmentation. This task involves pixel-level segmentation of optic disk. For this task, the disclosed embodiments use the Drishti-GS dataset, which includes 101 fundus images with expert-provided segmentation masks. The disclosed embodiments follow the official split, using 50 images for training and 51 for testing. Mean Dice score is used to evaluate optic disk segmentation.

### C. Benchmarking Adam-v2

For a comprehensive evaluation, in addition to training from scratch (the lower bound baseline), Adam-v2 is compared with a representative set of eight SOTA public self-supervised methods. These methods leverage ConvNet and vision transformer backbones and encompass diverse training objectives at the instance, patch, and pixel levels. Among them, PCRLv2, DiRA, and Adam-v1 are state of the art (SOTA) methods specifically tailored for medical tasks. Additionally, Adam-v2 is compared with publicly available and official large-scale models, including the RadImageNet, pretrained on 1.3 million medical images in a fully supervised manner, as well as Medical-MAE, RAD-DINO, and LVM-Med, pretrained on 510,000, 838,336, and 1.3 million medical images, respectively, via self-supervision. The disclosed embodiments use the backbone of the pretrained teacher of Adam-v2 (i.e., g_{θ}_{_{T}}) for feature analysis, few-shot transfer, and full transfer evaluations. In transfer learning to classification tasks, the disclosed embodiments use Adam-v2's pretrained backbone and concat a fully connected layer to generate the task-specific classification outputs. The disclosed embodiments use Adam-v2 base in anatomy understanding evaluations and ablation studies. In transfer learning to segmentation tasks, the disclosed embodiments employ a U-Net architecture, initializing the encoder weights with Adam-v2's pretrained backbone. Additionally, for the Adam-v2 large model, the disclosed embodiments adhere to the official segmentation network for the ConvNeXt backbone. Following the standard protocol, the disclosed embodiments perform end-to-end fine-tuning of all parameters of the downstream models. The disclosed embodiments strive to optimize each downstream task using the most effective hyperparameters. In classification tasks with ResNet/ConvNeXt backbones, the disclosed embodiments use the AdamW/Adam optimizer with a learning rate of 2.5e-4/1e-4, (β₁, β₂)=(0.9, 0.95) (0.9, 0.999), and weight decay of 0.05/0, and decrease the learning rate with a cosine/ReduceLROnPlateau scheduler. In segmentation tasks, the disclosed embodiments use the Adam optimizer with a learning rate of 1e-3 for the VinDR-Ribs, DRIVE, and Drishti-GS datasets, and the AdamW optimizer with a learning rate of 2e-4 for the rest of the tasks. The disclosed embodiments use the cosine learning rate decay scheduler and standard data augmentations, including random crop, brightness contrast, grid and optical distortion, elastic transformation, and gamma adjustment. Furthermore, the disclosed embodiments employ early stopping using 10% of the training data as the validation set. The disclosed embodiments run each downstream model at least five times and report statistical analysis using an independent two-sample t-test.

### IV. Results

This section outlines the cornerstones of results, with the goal of demonstrating that the learned representation of Adam-v2 is (a) semantics-rich, possessing unique properties essential for understanding anatomy and (b) versatile, showing strong generality and adaptability for a wide range of downstream tasks. To showcase the anatomy understanding capabilities of the framework, the disclosed embodiments delve into the unique learned properties (§ IV-A) and emergent properties (§ IV-B) of Adam-v2's embeddings (Eve-v2) via designing and conducting a set of experiments without adaptation (no finetuning) on various tasks, and the disclosed embodiments show: (i) Eve-v2 preserves localizability of anatomical structures (§ IV-A1), (ii) Eve-v2 preserves composability & decomposability of anatomical structures (§ IV-A2), (iii) Eve-v2 understands anatomical layouts via interpolation & extrapolation (§ IV-B1), (iv) Eve-v2 possesses rich dense anatomical features (§ IV-B2), (v) Eve-v2 recognizes anatomical symmetries (§ IV-B3), (vi) Eve-v2 balances anatomical harmony and diversity (§ IV-B4), and (vii) Eve-v2 showcases robust anatomical structure matching (§ IV-B5). To demonstrate the versatility of the framework's learned representation, the disclosed embodiments assess the robustness and generalizability of Adam-v2's representation through a series of extensive experiments via adaptation (fine-tuning) on myriad tasks, and the disclosed embodiments show: (i) Adam-v2 excels in few-shot transfer (§ IV-C) and (ii) Adam-v2 stands out in full transfer (§ IV-D).

### A. Learned Properties

This section investigates the learned properties of Adam-v2's embeddings, Eve-v2, which were imposed by each of the three branches (see FIG. 3) during training in the self-supervised learning framework (§ II).

### 1) Learned Property 1: Eve-v2 Preserves Localizability of Anatomical Structures:

Experimental setup. Adam-v2's capability to differentiate various anatomical structures was examined to ascertain if the learned embeddings (Eve-v2) preserve the locality of anatomical structures. To do so, the disclosed embodiments compile a dataset of 1,000 images (from the ChestX-ray14 dataset) with 10 distinct anatomical landmarks manually marked by human experts in each image. With reference to FIG. 4, Eve-v2 preserves localizability of anatomical structures, effectively distinguishing different anatomical landmarks. As seen, the baselines fall short in generating distinct features for different anatomical landmarks, leading to ambiguous embedding spaces with mixed clusters. In contrast, Adam-v2 effectively discriminates between various anatomical landmarks, resulting in well-separated clusters within its learned embedding space. The disclosed embodiments extract patches of size 224² around the location of each landmark across images and derive latent features (i.e., embeddings) of each landmark instance using each pretrained model under study (without fine-tuning).

The disclosed embodiments visualize the embeddings by projecting them onto a 2-dimensional space using t-SNE. The disclosed embodiments compare Adam-v2 with a representative selection of supervised and SSL methods.

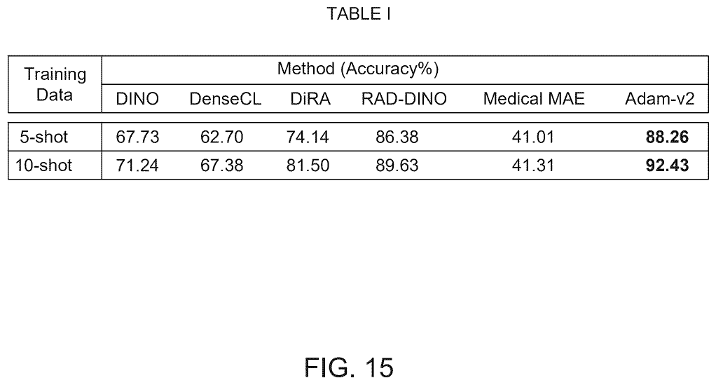

Results. As seen in FIG. 4, Adam-v2 effectively distinguishes different anatomical structures from one another, resulting in well-separated clusters within its learned embedding space. In contrast, the baseline methods fail to provide distinguishable features for different anatomical structures, producing ambiguous embedding spaces with mixed clusters. The disclosed embodiments complement the qualitative results (t-SNE plots) with quantitative analysis by evaluating the accuracy of each pretrained model in classifying anatomical structures via training a linear classifier on top of each model in a few-shot setting. Specifically, each model was trained with 5 and 10 samples (shots) per class and subsequently evaluated on the test data. As seen in Table I presented in FIG. 15, Adam-v2 shows superior accuracy in classifying instances of each class, outperforming the runner-up baseline by 1.9% and 2.8% for the 5-shot and 10-shot settings, respectively. In summary, considering both qualitative and quantitative results, Adam-v2 effectively preserves the localizability of anatomical structures within its embedding space (Eve-v2).

### 2) Learned Property 2: Eve-v2 Preserves Composability & Decomposability of Anatomical Structures:

Experimental setup. The disclosed embodiments explore Adam-v2's ability to capture part-whole hierarchies to determine if the learned embeddings (Eve-v2) preserve the composability and decomposability of anatomical structures—the embedding of a whole should be equal or close to the sum of the embedding of its each part (see E(P) examples in FIG. 5A). To do so, the disclosed embodiments extract random patches of varying sizes, called whole, from 1,000 images chosen randomly from the test images of ChestX-ray14 dataset. Each whole is decomposed into 2, 3, or 4 non-overlapping parts with different sizes. The disclosed embodiments resize each whole and its parts to 224², extract their features using pretrained models under study, and compute the cosine similarity between the embedding of each whole and the aggregate of its parts. The disclosed embodiments visualize the similarity distributions with the violin plot.

Results. As seen in FIG. 5B, the violin plot reveals that the median similarity for Adam-v2 is notably higher than other SSL baselines. Furthermore, the distribution of similarity values for Adam-v2 is prominently centered around the 1.5× interquartile, positioned at the upper end of the violin plot. This concentration implies that, in the majority of cases, the similarity value between the embedding of entire wholes and their aggregated parts is closer to 1 in the Adam-v2 model.

### B. Emergent Properties

This section explores the emergent properties of Adam-v2's learned embeddings, Eve-v2, which were not explicitly targeted during training but have naturally developed.

### 1) Emergent Property 1: Eve-v2 Understands Anatomical Layouts Via Interpolation & Extrapolation:

Experimental setup. To showcase the proficiency of Adam-v2 in understanding anatomy, the disclosed embodiments investigate its capability to interpolate/extrapolate embeddings for a randomly selected anatomical structure, utilizing the embeddings of two other randomly chosen anatomical structures. For the interpolation, two random source coordinates (denoted as A and B in FIG. 6A) are chosen, and the established interpolation formula (see FIG. 6B) is applied to generate a random point C. Specifically, patches of size 224² are extracted around points A, B, and C, passed through each pretrained model under study to extract their respective embeddings E_{A}, E_{B}, and E_{C}, with E_{C} serving as the ground truth for evaluating the interpolated embeddings for C. Following that, the interpolation formula is applied using E_{A} and E_{B} to generate embeddings for C, yielding interpolated embeddings E′_{C}. Finally, the cosine similarity between the interpolated embeddings E′_{C} and the ground truth E_{C} is computed. The process was repeated for 1,000 images chosen randomly from the test images of ChestX-ray14, utilizing three different values of t₁ (i.e., t₁∈{0.25, 0.5, 0.75}). Box plots are used to illustrate the similarity distributions in each setting. Notably, the extrapolation of embeddings for a randomly selected point D is examined in a similar manner using the standard extrapolation formula.

Results. As seen in FIG. 6B, the boxplots reveal the consistent advantage of the Adam-v2 model in achieving higher similarity between interpolated/extrapolated embeddings and the ground truth compared with other baselines. Specifically, across various values of t₁ for interpolation and t₂ for extrapolation, Adam-v2 consistently outperforms other baselines, delivering a median similarity close to 1. This remarkable performance highlights the effectiveness of Adam-v2 in establishing precise layout relationships between anatomical structures. It is noteworthy that the Adam-v2 model was not explicitly trained to learn these two properties (interpolation & extrapolation). However, their emergence highlights the capabilities of Adam-v2 in understanding anatomy.

### 2) Emergent Property 2: Eve-v2 Possesses Rich Dense Anatomical Features:

Experimental setup. To showcase Adam-v2's effectiveness in producing rich dense embeddings, the disclosed embodiments explore its representations (Eve-v2) for zero-shot co-segmentation of common anatomical structures across patients. The disclosed embodiments use Adam-v2's pretrained backbone to extract spatial dense descriptors and saliency maps from a set of random chest radiographs. The descriptors are then clustered, with each cluster classified as foreground or background based on saliency map voting. The identified foreground clusters form the co-segmentation results.

Results. As shown in FIG. 7, Adam-v2 transforms each pixel in chest radiographs into semantically meaningful embeddings (Eve-v2), forming multiple “echo chambers” (produced via co-segmentation. Indeed, without fine-tuning, Adam-v2 co-segments common anatomical structures across different patients-distinct anatomical structures are associated with unique dense embeddings (illustrated by distinct colors in FIG. 7), while the same anatomical structures exhibit nearly identical embeddings across patients. This observation highlights Adam-v2's ability to understand anatomy, as Eve-v2 possesses rich dense anatomical features.

### 3) Emergent Property 3: Eve-v2 Recognizes Anatomical Symmetries:

Experimental setup. To scrutinize the effectiveness of Adam-v2 in comprehending anatomy, the disclosed embodiments examine its ability to recognize the symmetry of anatomical structures-mirrored anatomical structures (e.g., right clavicle and left clavicle) should be associated with mirrored embeddings. To do so, the disclosed embodiments consider four pairs of mirrored anatomical structures (as marked in FIG. 8A). The disclosed embodiments extract 224² patches around each anatomical structure marked with a “symbol” in the images and derive embeddings for each instance using the Adam-v2 pretrained model. For the mirrored anatomical structures, the disclosed embodiments extract 224² patches around each anatomical structure marked with a “cross” (mirrored counterparts of the cross-marked anatomical structures), flip these patches, and then derive embeddings for each flipped instance using the Adam-v2 pretrained model. The disclosed embodiments visualize the embeddings by projecting them onto a 2-dimensional space using t-SNE.

Results. As shown in FIG. 8B, four distinct clusters appear in the embedding space of Adam-v2. Within each cluster, the embeddings of each pair of mirrored anatomical structures overlap, forming cohesive groups that are distinguishable from other clusters. This observation highlights the ability of Adam-v2 to understand the symmetry of anatomical structures within its learned embedding space. It is noteworthy that during the pretraining of Adam-v2, image flipping was not used as an augmentation technique, nor was the model explicitly trained to learn this property. This property's emergence further highlights Adam-v2's proficiency in comprehending anatomy.

### 4) Emergent Property 4: Eve-v2 Balances Anatomical Harmony and Diversity:

Experimental setup. The disclosed embodiments evaluate Adam-v2's ability in securing consistent embeddings for similar anatomical structures across different scales, while also encoding nuanced diversity of intensity, shape, boundary, and texture among instances of the same anatomical structure across patients, aiming to determine if the learned embedding space balances anatomical harmony and diversity. To do so, the disclosed embodiments randomly select four distinct anatomical landmarks, and extract three patches at different scales, (labeled as levels 1, 2, and 3) around each landmark across the images of different patients (see FIG. 9A).

The disclosed embodiments then compute the embeddings of these patches using Adam-v2's pretrained model and visualize them with a t-SNE plot. The disclosed embodiments also provide zoomed-in sub-plot for a landmark class, where the embeddings of patches at different “scales” are represented by circles, triangles, and crosses in the feature space; the embeddings of patches from different “patients” are distinguished by different shapes.

Results. As seen in FIG. 9B, for each of the four different landmarks (i.e. anatomical structures), their embeddings at levels 1, 2, and 3 are closely clustered, demonstrating Adam-v2's capability to maintain harmony in embeddings of semantically similar anatomical structures across scales and patients. Moreover, for each landmark, embeddings of patches at different scales for the same patient are closely grouped, while those from different patients are distinctly separated, showing Adam-v2's ability to preserve the diversity of anatomical structures across different patients. To quantitatively verify this observation, the disclosed embodiments design and conduct two experiments:

(1) Within-vs-between patients' classification: The disclosed embodiments select a random anatomical structure and create a dataset with positive and negative samples. Positive samples are formed by concatenating embeddings of patches at scales 1, 2, and 3 from the same patient. Negative samples are formed by concatenating embeddings of patches at scales 1, 2, and 3, each randomly selected from different patients. Using this dataset, the disclosed embodiments train a linear classifier to predict whether a set of three concatenated embeddings belongs to patches from the same patient or not. Adam-v2 achieved a 96.2% AUC, demonstrating its robust capability to distinguish between patches from the same patient and those from different patients.

(2) Anatomical structure retrieval: The disclosed embodiments select a random anatomical structure and search for the nearest neighbors of each instance (query) of the selected anatomical structure in the latent space across different scales and patients. For each patch at scale 1, the disclosed embodiments find its nearest neighbors in the embedding space among all patches at scale 2 across all patients. If a patch at scale 2 from the same patient is among the retrieved patches, the retrieval is considered a correct prediction. The disclosed embodiments calculate the top 5 retrieval accuracy by dividing the number of correct predictions by the total number of patients. The disclosed embodiments repeat this process for scale 1 to 3 and scale 2 to 3 and report the average top 5 accuracy across all three combinations. Adam-v2 achieved a 97.6% top 5 accuracy, confirming its superior retrieval capabilities in identifying and associating relevant patches from the same patient across varying scales.

In summary, these qualitative and quantitative results show Adam-v2's ability to distinguish embeddings between patients while providing consistent embeddings for the same patients.

### 5) Emergent Property 5: Eve-v2 Showcases Robust Anatomical Structure Matching:

Experimental setup. To further illustrate Adam-v2's capability in anatomy understanding, the disclosed embodiments examine robustness of Adam-v2's representations (Eve-v2) for anatomical structure matching in diverse scenarios. To do so, from a given query image, the disclosed embodiments randomly select N_{q}=13 anatomical structure and extract a patch of size 96² centered at each anatomical structure. These patches are resized to 224² and passed through Adam-v2's pretrained backbone to generate query embeddings

$${E}_{q} = {\{ {E}_{q}^{i} \}}_{i = 1}^{{N}_{q}} .$$

Then, for an unlabeled key image, the disclosed embodiments extract Nₖ patches by sliding a window of size 96² with a stride of 16 over the image. After resizing these key patches to 224², their embeddings

$${E}_{k} = {\{ {E}_{k}^{j} \}}_{j = 1}^{{N}_{k}^{-}}$$

re-obtained using Adam-v2's pretrained backbone. Finally, for each query anatomical structure embedding in E_{q}, the disclosed embodiments compute its f₂-distance with all embeddings in Eₖ and identify the center of the patch corresponding to the embeddings with the minimum distance as the matched point for the anatomical structure. To showcase the robustness of Eve-v2 to anatomical variations, the disclosed embodiments explore three distinct settings involving anatomical structure matching across images of the same patient with different diseases, images of different patients, and augmented views of the same image. FIG. 10 depicts the query image annotated with 13 anatomical structures (squares) and corresponding matched points (indicated by crosses) across different diseases, patients, and views. To assess the accuracy of matched points, the disclosed embodiments include the ground truth anatomical structure provided by human experts for key images (depicted as squares).

Results. As shown in Figure FIG. 10, Eve-v2 showcases its potential in precisely identifying similar anatomical structures, demonstrating robust performance in matching anatomical structures in images of the same patient with different diseases, across different patients, and in augmented views of the same image. These observations emphasize the semantic richness inherent in Adam-v2's representations and underscore an additional emergent property of Adam-v2.

### C. Adam-v2 Excels in Few-Shot Transfer

Experimental setup. The disclosed embodiments investigate Adam-v2's potential as a robust foundation for fine-tuning deep models in limited data regimes, aiming to demonstrate the strength of its learned representations in the absence of large-scale labeled data. To do so, the disclosed embodiments compare Adam-v2 with publicly available and official fully/self-supervised models (e.g., Medical-MAE, RAD-DINO) using limited labeled data for segmentation tasks involving anatomical structures (i.e., heart and clavicle) and various thoracic diseases (e.g., pneumothorax, emphysema, fibrosis). Specifically, the disclosed embodiments fine-tune the pretrained models with a few labeled data shots (3, 6, 12, and 24) randomly sampled from the JSRT-Clavicle and JSRT-Heart datasets, as well as two randomly selected limited label fractions (5% and 10%) from the SIIM-ACR and ChestX-Det datasets.

Results. As seen in Table II presented in FIG. 16, Adam-v2 shows superior few-shot transfer performance over baselines across all label fractions for both anatomical structure and disease segmentation tasks: (1) In clavicle segmentation, Adam-v2 surpasses the runner-up by 23.6%, 7.81%, 2.83%, and 3.13% for 3, 6, 12, and 24 shots, respectively. For heart segmentation, Adam-v2 achieves higher performance with differences of 3.94%, 5.04%, 2.59%, and 0.45% for 3, 6, 12, and 24 shots, respectively. In disease segmentation tasks, with 5% and 10% labeled data subsets, Adam-v2 surpasses the runner-up by 8.85% and 3.37% for the pneumothorax segmentation task in the SIIM-ACR dataset, and by 0.97% and 0.18% for the thoracic disease segmentation task in the ChestX-Det dataset, respectively. (2) Adam-v2 (base model) outperforms Adam-v1 across all label fractions in all tasks, showing the effectiveness of the methodological improvements. Also, Adam-v2 surpasses Adam-v2 (base model) across all label fractions in all tasks, demonstrating the benefits of data scaling.

### D. Adam-v2 Provides Generalizable Representations for a Variety of Downstream Tasks

Experimental setup. The disclosed embodiments examine the generalizability of Adam-v2's representations through transfer learning across a wide range of downstream tasks in a full fine-tuning setting. To do so, the disclosed embodiments compare Adam-v2 with various SOTA ConvNet- and vision transformer-based SSL methods designed for medical applications. The disclosed embodiments also include training downstream models from random initialization (the lower-bound baseline) and the fully supervised RadImageNet model.

Results. As seen in Table III presented in FIG. 17, Adam-v2, across all tasks, consistently outperforms fully-supervised models and shows significant performance improvements (p<0.05) over all SSL counterparts. Moreover, Adam-v2 exceeds its base version by a large margin, demonstrating the efficiency of the framework for large-scale pretraining. The consistent performance gains over baseline methods underscore the versatility of the learned representations across various applications.

### V. Ablation Studies

This section presents the results of ablation studies. First, the disclosed embodiments explore the impact of including or excluding negative samples in the localizability branch for learning discriminative anatomical representations (§ V-A). Next, the disclosed embodiments examine the impact of the three learning branches of the framework (§ V-B).
the disclosed embodiments investigate the effect of the coarse-to-fine learning strategy (§ V-C). The disclosed embodiments perform an ablation study to assess the resilience of the framework to gender-imbalanced data (§ V-D). Also, the disclosed embodiments evaluate the efficacy of Adam-v2 for disease localization in a weakly-supervised setting (§ V-E). Finally, the disclosed embodiments show the generalizability of the framework to other imaging modalities by pretraining it on fundus images (§ V-F).

### A. Impact of Whether to Use Negative Samples

Experimental setup. The Adam-v2 framework uses self-distillation loss—which maximizes the similarity between positive pairs and eliminates the need for using negative pairs—as the default choice for its localizability branch (Eq. 5). Another widely used similarity loss function that can be employed in the localizability branch is InfoNCE loss—which simultaneously pulls positive pairs closer together while pushing negative pairs apart. The primary difference between these two types of similarity loss functions is the use of negative samples. To investigate the necessity of negative pairs for learning discriminative anatomical representations, the disclosed embodiments conduct an ablation study on the training objective of the localizability branch by training it via contrastive loss. The disclosed embodiments compare the resulting model's performance in both anatomical landmark discrimination and transfer learning to downstream tasks against its self-distillation counterpart.

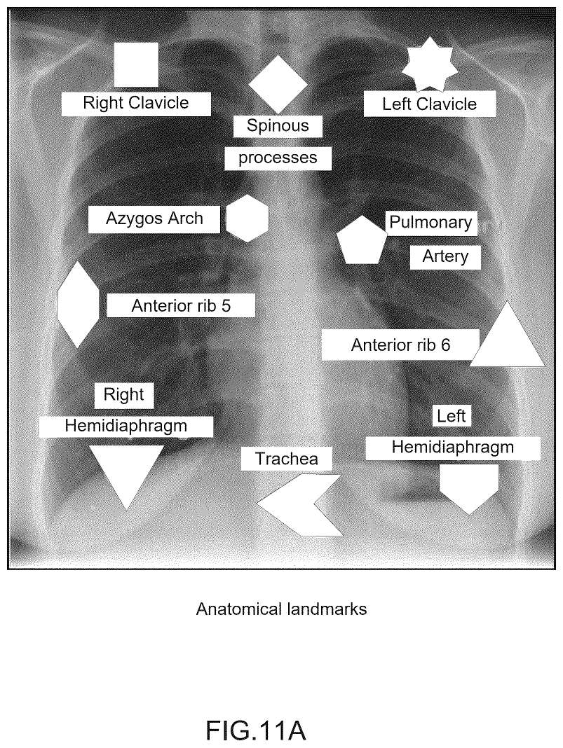

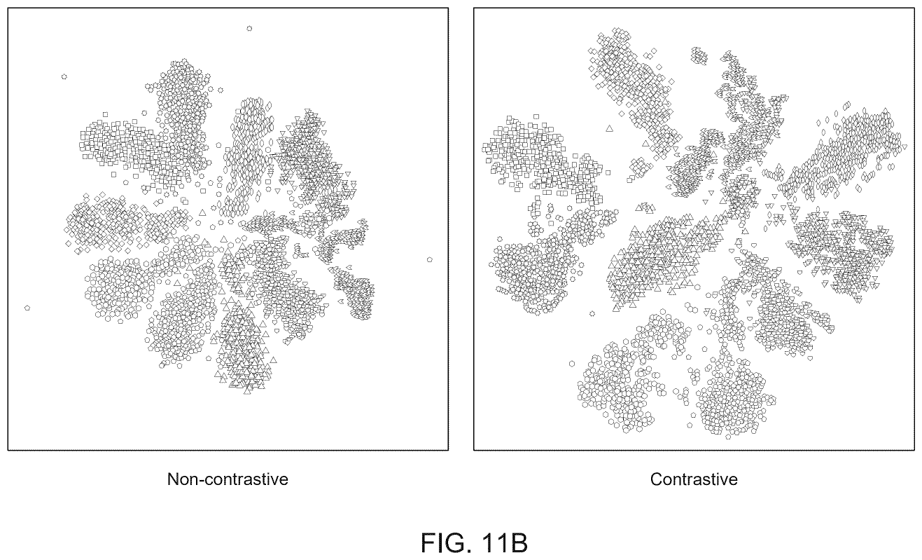

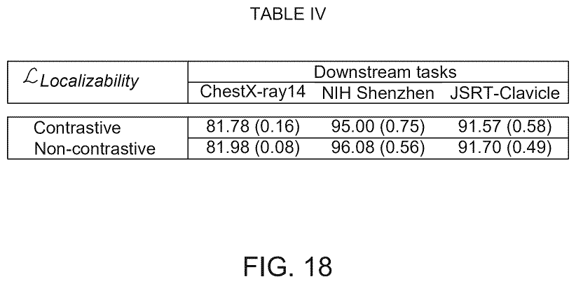

Results. As seen in FIGS. 11A and 111B, regardless of whether negative pairs are used in the localizability branch, the framework can learn a semantically structured embedding space where similar anatomical structures are clustered together and distinguished from dissimilar structures. Also, as shown in Table IV presented in FIG. 18, using self-distillation loss in the localizability branch yields slightly higher transfer performance in the target tasks than employing InfoNCE loss. Given these observations, self-distillation appears to be a more suitable choice than InfoNCE loss. The primary reason for opting for self-distillation loss over InfoNCE loss in the localizability branch is its computational efficiency, as it eliminates the need for negative pairs and facilitates efficient large-scale pretraining of Adam-v2 model.

### B. Impact of Learning Objectives

Experimental setup. Adam-v2 leverages three branches in representation learning: localizability, composability, and decomposability. To evaluate the impact of each branch, the disclosed embodiments conduct an ablation study, starting with the localizability branch and incrementally adding composability and decomposability. Each model is fine-tuned and evaluated on tuberculosis classification and thoracic disease segmentation tasks.

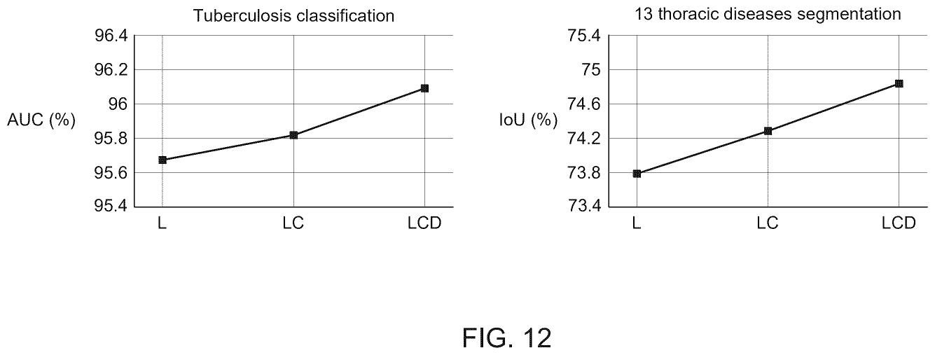

Results. As seen in FIG. 12, adding the composability branch to the localizability branch consistently enhances performance across tasks. Furthermore, incorporating the decomposability branch along with these two branches results in even greater improvements, leading to significant performance boosts (p<0.05) in both tasks compared with using the localizability branch alone. In summary, integrating these three branches into a unified self-supervised learning framework yields representations with higher generality and transferability, resulting in powerful Adam-v2 models that can effectively serve as the foundation for numerous target tasks through fine-tuning.

### C. Impact of Coarse-to-Fine Learning

Experimental setup. The Adam-v2 framework leverages coarse-to-fine learning strategy that integrates diverse anatomical structures at various scales to develop hierarchical representations. To investigate the impact of hierarchical learning of anatomical structures at various scales (i.e. m) in a coarse-to-fine manner, the disclosed embodiments conduct an ablation study. To do so, the disclosed embodiments start with m=0, where the model is trained on the entire anatomy (whole images). The disclosed embodiments then progressively reduce the scale of anatomical structures by factors based on powers of 2, delving deeper into the higher levels of the anatomical hierarchy to represent finer structures.

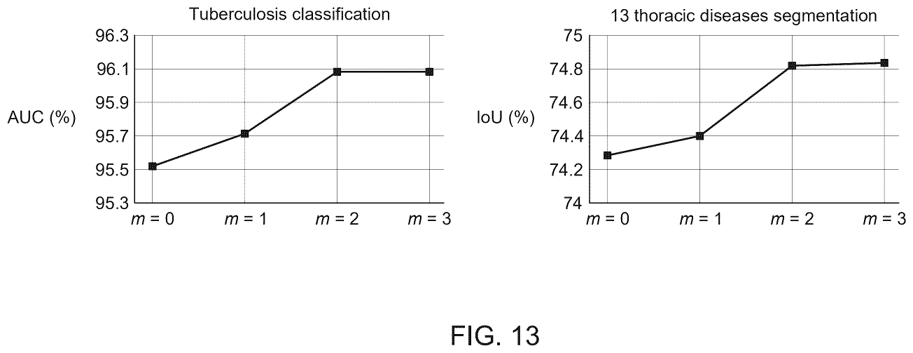

Results. As seen in FIG. 13, gradually increasing data granularity from m=0 to m=2 consistently enhances downstream performance. This demonstrates that the coarse-to-fine learning strategy incrementally deepens the model's anatomical knowledge, leading to more generalizable representations for various tasks. Furthermore, there is no significant performance change at m=3, suggesting that pretraining up to level 2 provides sufficiently robust representations.

### D. Gender Bias Analysis

Experimental setup. A robust model should demonstrate a high level of resistance to biased training data, particularly regarding gender biases, as structural variations in images between genders—male to female (or vice versa)—can cause shifts in data distribution. Gender bias is evident when there is a notable decrease in performance when training and test data involve different genders, compared to when they involve the same gender. To evaluate the robustness of the learned representations through the framework to gender biases, the disclosed embodiments perform an ablation study assessing the impact of gender imbalance on the transfer performance of Adam pretrained models, including Adam-v1, Adam-v2 (base), and Adam-v2. To do so, the disclosed embodiments use the train/test splits for the ChestX-ray14 dataset, ensuring a balanced number of cases per class in both the male-only and female-only folds. The disclosed embodiments fine-tune the Adam models using each male-only and female-only training fold and then evaluate them on the corresponding male-only and female-only test folds. This process is repeated for all folds, and the disclosed embodiments report the average performance across all folds. The disclosed embodiments assess the AUC difference on a test split consisting of only one gender by comparing models trained on the same gender versus those trained on the opposite sex.

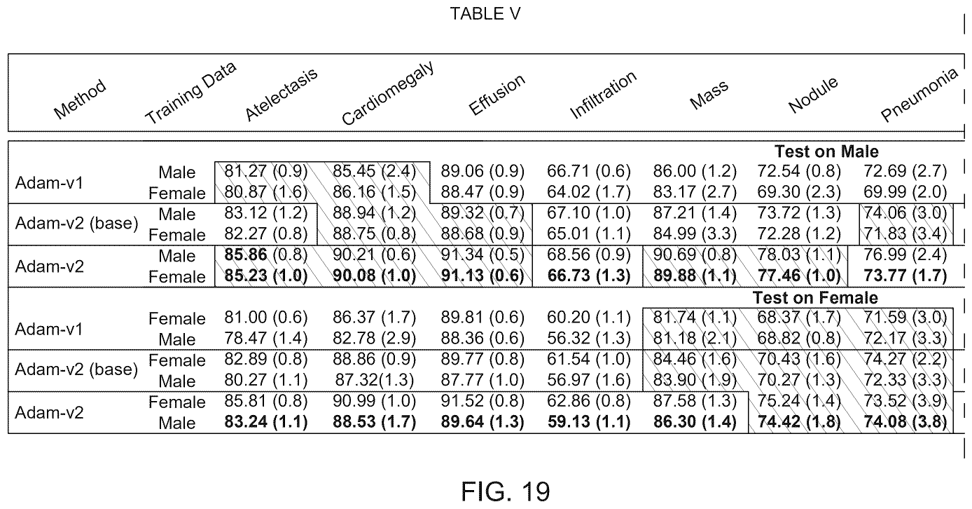

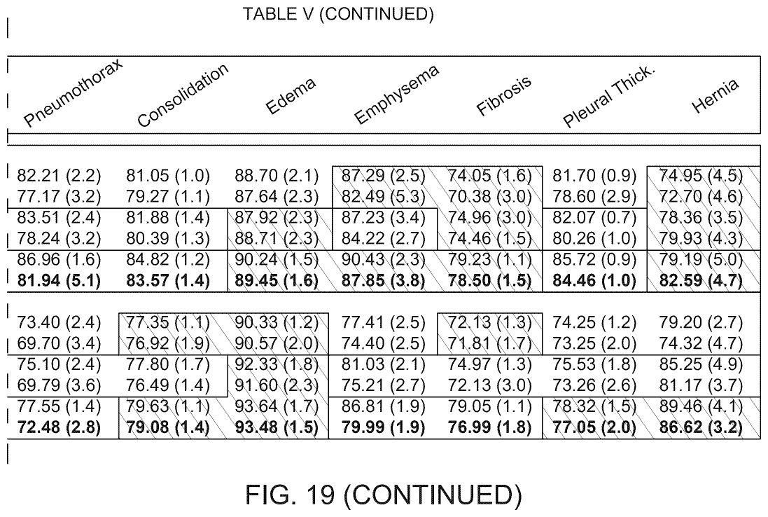

Results. As seen in Table V presented in FIG. 19, Adam-v2 demonstrated less biased performance compared with both Adam-v1 and Adam-v2 (base). Specifically, Adam-v2 consistently outperforms the other models across all diseases when the training and testing data come from opposite genders. For instance, when fine-tuned on female-only patients and tested on male-only patients, Adam-v2 achieves an average performance improvement of 5.2% over Adam-v1 and 3% over Adam-v2 (base). Moreover, when the models were fine-tuned on female-only patients and tested on male-only patients, Adam-v1 and Adam-v2 (base) exhibited unbiased performance for 5 and 6 out of 14 diseases, respectively, whereas Adam-v2 demonstrated unbiased performance for 9 diseases. Additionally, when the models were fine-tuned on male-only patients and tested on female-only patients, Adam-v1 and Adam-v2 (base) showed unbiased performance for 6 and 4 diseases, respectively, whereas Adam-v2 maintained unbiased performance for 6 diseases, along with a notable performance boost across all diseases compared to both baseline models. These findings indicate that Adam-v2, in comparison to its base version and the earlier Adam-v1, displays enhanced robustness to the highly imbalanced data that leads to gender bias in medical image analysis.

### E. Weakly-Supervised Disease Localization Analysis

Experimental setup. The disclosed embodiments evaluate the efficacy of Adam-v2 for localizing chest pathologies in a weakly supervised setting using the ChestX-ray14 dataset, which includes 787 cases annotated with bounding boxes for eight thoracic diseases. During training, the disclosed embodiments fine-tune Adam-v2 and other baselines for thoracic disease classification tasks using only image-level disease labels. For testing, the disclosed embodiments apply Grad-CAM to visualize the regions of the image that contribute to the predictions of the model, specifically identifying the diseased areas.

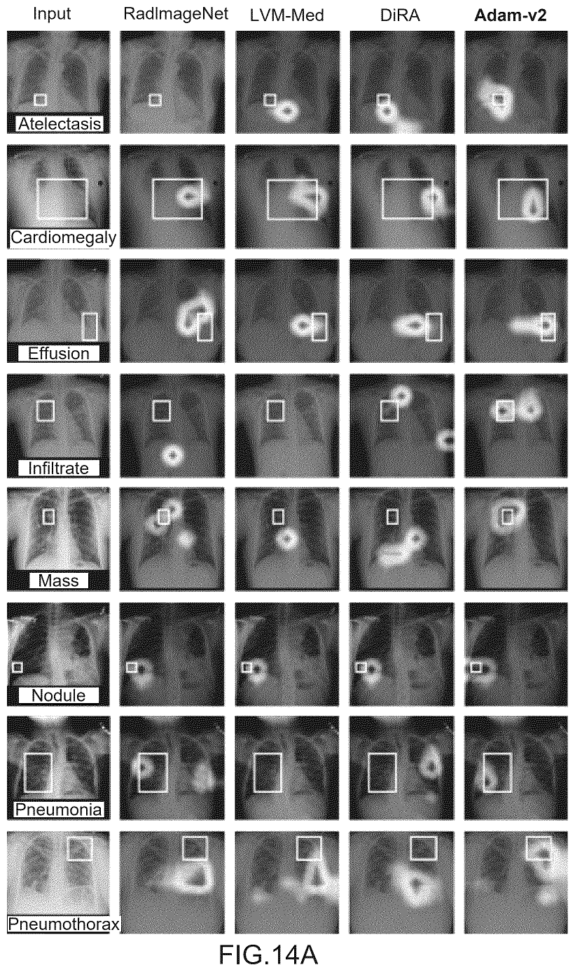

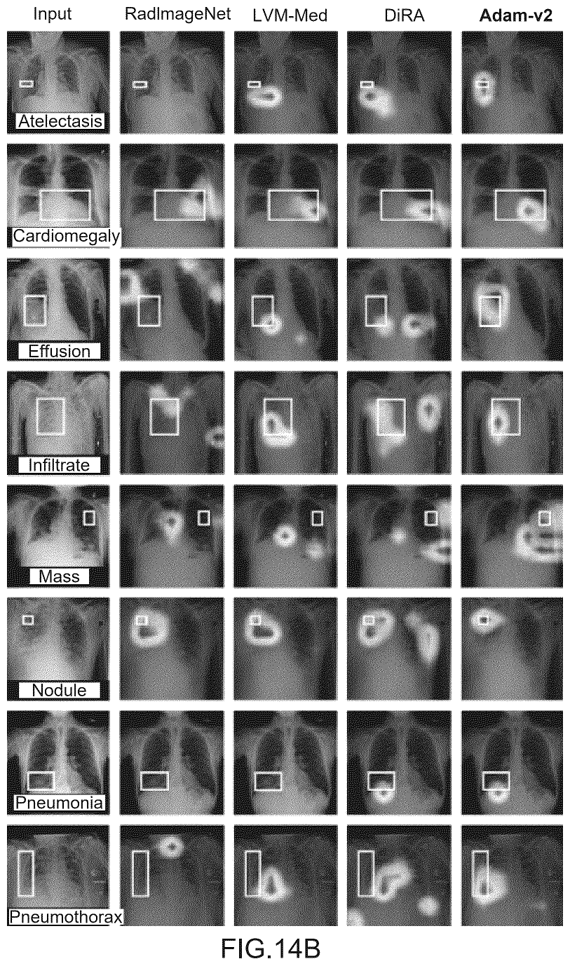

Results. FIGS. 14A and 14B depict the visual interpretation of thoracic diseases diagnoses using Grad-CAM across eight different pathologies. As seen, Adam-v2 achieves more accurate disease localization compared to other baselines. Notably, the heatmaps generated by Adam-v2 demonstrate a greater concentration around disease regions, with its attention maps exhibiting a more pronounced overlap with the ground truth across all diseases. This generation of more interpretable activation maps not only highlights the Adam-v2's potential for precise disease localization but also shows its potential for clinical utility in post-hoc interpretation by radiologists.

### F. Generalizability of the Framework

Experimental setup. The framework can be adapted to other imaging modalities. To show this, the disclosed embodiments pretrain Adam-v2 on fundus images and subsequently assess its performance on fundus-related segmentation tasks via transfer learning in different settings. Specifically, the disclosed embodiments pretrain Adam-v2 on the EyePACS dataset, then fine-tune it for retinal vessel segmentation on the DRIVE dataset and optic disk segmentation on the Drishti-GS dataset, considering both few-shot and full fine-tuning settings. The Adam-v2 model and baseline models, all pretrained on the EyePACS dataset, are fine-tuned with a limited number of labeled samples (3-shot, 6-shot, 12-shot) and the full set from the DRIVE and Drishti-GS datasets.

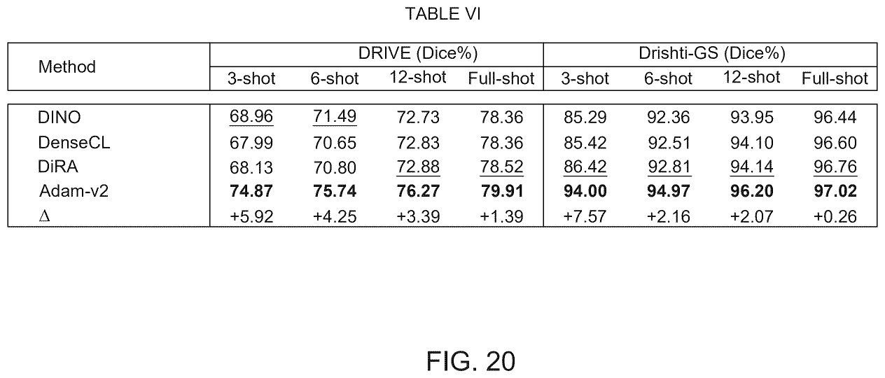

Results. As seen in Table VI presented in FIG. 20, Adam-v2 exhibits superior performance (p<0.05) across fundus-related segmentation tasks in both few-shot and full transfer settings. Specifically, in retinal vessel segmentation on the DRIVE dataset, Adam-v2 outperforms the runner-up baseline, achieving improvements of 5.9%, 4.2%, and 3.3% in the 3-shot, 6-shot, and 12-shot settings, respectively. Similarly, in optic disk segmentation on the Drishti-GS dataset, Adam-v2 provides superior performance compared with the runner-up baseline, with improvements of 7.6%, 2.2%, and 2.1% in the 3-shot, 6-shot, and 12-shot settings, respectively. Notably, with only 3-shot settings, Adam-v2 achieves 94% of its full training data performance in the retinal vessel segmentation task and 97% in the optic disk segmentation task.

### VI. Related Works

### A. Self-Supervised Learning

Self-supervised learning (SSL) methods aim to learn general representations without human supervision. Numerous studies focus on learning global features by discriminating data samples at either the cluster level or the instance level. Clustering methods group the data into clusters via a similarity metric, and train SSL models to distinguish these clusters. On the other hand, instance discrimination methods compel the SSL models to generate similar features for different views of the same image, employing various strategies to prevent trivial solutions. Contrastive methods use negative samples to discriminate between images. Self-distillation methods bypass the need for negative pairs by training a student network to predict the features of a teacher network. Feature decorrelation methods maximize the information content of the representations while also enforcing similarity between instances of the same image. Notably, recent works have explored instance discrimination for pretraining vision transformers (ViT).

Alternatively, dense SSL methods focus on learning local features by encoding visual patterns embedded at smaller image regions. Several studies have delved into dense contrastive learning, aiming to mine correspondences between pixels at the same spatial location, similar pixels or patches in a feature map, or similar image regions. In addition, masked image modeling methods mask/perturb random parts of the input image and reconstruct the missing parts at pixel-level.

Inspired by recent advancements in SSL for computer vision tasks, there has been a surge in its application to medical imaging tasks. Recent studies focus on instance discrimination and image reconstruction pretext tasks, as well as their integration. These advancements have enabled SSL to become the cornerstone for developing broadly applicable foundation models. However, current SSL methods often neglect anatomical hierarchies in their learning objectives, resulting in limited anatomy understanding capabilities. By contrast, Adam-v2 leverages the hierarchical nature of anatomy to learn semantically rich features, yielding more powerful models for a broad variety of medical tasks.

B. Learning from Anatomy

The consistent structure in medical images harbors rich semantics about human anatomy, thereby providing strong supervision signals for deep semantic representation learning via self-supervision. Existing self-supervised methods focus on reconstructing anatomical patterns from transformed images, learning semantics of consistent anatomical patterns across patients with subsequent enhancements via adversarial learning, exploiting spatial relationships in anatomy, utilizing global and local anatomical consistency, and incorporating anatomical cues to improve contrastive learning. These existing works neglect hierarchical anatomy relations. While the earlier method Adam-v1 uses anatomy hierarchies as soft supervisory signals, Adam-v2 explicitly captures part-whole hierarchies via its learning objectives. Compared with Adam-v1, Adam-v2 offers two significant advancements: (1) enhancing the localizability branch by eliminating negative pairs, thereby improving computational efficiency for large-scale pretraining, (2) introducing two novel components: composability and decomposability, which are crucial for capturing part-whole hierarchies.

### C. Learning Part-Whole Hierarchies

Learning hierarchical representations has been embedded in deep architectures such as ConvNets and hierarchical vision transformers (ViT). However, the multi-scale feature hierarchy learned by conventional neural networks does not explicitly align with the natural part-whole relationships in visual data. This limitation has prompted the development of new architectures for modeling part-whole hierarchies. Notably, GLOM introduced a conceptual framework that leverages attention to explicitly learn part-whole relationships, and recent works have proposed ViT-based architectures to implement it. In contrast to them, Adam-v2 moves beyond architecture design by devising a novel learning strategy that encodes the semantics of part-whole hierarchies into the embedding space through three explicit training objectives: localizability, composability, and decomposability.

### D. Previous Work

The inventors first introduced a preliminary version of Adam-v2, a novel self-supervised learning strategy that captures inherent hierarchical relationships within medical images, in the CVPR 2024 publication. The disclosed embodiments significantly extend the preliminary version with the following improvements.
1) The disclosed embodiments have introduced notations, diagrams, and additional technical details throughout the paper for a succinct illustration of motivation, framework, and experiments.
2) The disclosed embodiments have examined Adam-v2 with recent baselines for anatomy understanding—including localizability, composability & decomposability, and interpolation & extrapolation of anatomical structures, demonstrating Adam-v2's superiority in producing semantics-rich anatomical embeddings.
3) The disclosed embodiments have performed quantitative evaluations on few-shot landmark classification, demonstrating the superiority of Adam-v2 in preserving the localizability of anatomical structures compared to existing SSL methods.
4) The disclosed embodiments have quantitatively evaluated Adam-v2's ability to balance anatomical harmony and diversity across two settings: within-vs-between patient classification and image retrieval.
5) The disclosed embodiments have designed new experimental suites to showcase additional emerging properties of Adam-v2, including its capability to produce rich, dense anatomical embeddings, recognize anatomical symmetries, and match corresponding anatomical structures across views/patients.
6) The disclosed embodiments have conducted extensive experiments to compare Adam-v2 models against an updated set of large-scale fully- and self-supervised baselines in few-shot transfer settings, highlighting the robustness of Adam-v2 in limited data regimes.
7) The disclosed embodiments have rigorously evaluated Adam-v2 models (base and large-scale) against an updated set of fully- and self-supervised baselines in full-shot transfer settings, proving the superiority of Adam-v2 across seven tasks.
8) The disclosed embodiments have conducted quantitative and qualitative ablation studies on different loss function choices for the localizability branch, highlighting the framework's flexibility in learning discriminative anatomical features with various loss functions.
9) The disclosed embodiments have performed an in-depth study on the impact of gender imbalance in the data, highlighting the robustness of Adam-v2 in mitigating gender biases.
10) The disclosed embodiments have evaluated Adam-v2 in few-shot settings for downstream tasks in the fundus modality, showcasing its label efficiency in tasks with extremely limited data.

### VII. Conclusion

The disclosed embodiments present Adam-v2, a self-supervised learning framework designed to enhance visual representations by constructing hierarchical embeddings for distinct anatomical structures. The core novelty of the approach is the explicit enforcement of part-whole hierarchies within an SSL framework. The experiments validate the effectiveness of Adam-v2 across diverse tasks, consistently outperforming multiple baselines. Furthermore, the disclosed embodiments demonstrate the semantic richness of the learned representations, which arise from both explicitly acquired and autonomously emerging unique properties.

Limitation: In the current implementation of Adam-v2, the disclosed embodiments decompose each random anchor (w) into four non-overlapping parts (p1-p4). As the training schema follows a coarse-to-fine manner, the size of w is halved at each training stage, leading to a proportional reduction in the size of its subparts. The ablation study (FIG. 13, m=2→m=3) reveals that performance saturates when the size of w decreases from ¼ to ⅛ of the original image resolution. While this suggests that breaking w into more than four parts may not significantly affect performance, it warrants further investigation.

Thus, the disclosed embodiments, referred to herein as Adam-v2, provide a self-supervised learning framework that explicitly learns to encode inherent part-whole hierarchies within medical images through three key branches: (1) Localizability, acquiring discriminative representations to distinguish different anatomical structures; (2) Composability, learning each anatomical structure in a parts-to-whole manner; and (3) Decomposability, comprehending each anatomical structure in a whole-to-parts manner. The extensive experiments described herein showcase Adam-v2's advanced capability in anatomy understanding, unveiled through its embeddings (Eve-v2). Particularly, Adam-v2 demonstrates a zero-shot understanding of anatomy as revealed through its learned properties: \textcircled{1} preserving localizability of anatomical structures and \textcircled{2} encoding part-whole relations of anatomical structures as well as its emergent properties: \textcircled{3} understanding anatomical layouts via interpolation and extrapolation, \textcircled{4} associating each image pixel with (dense) semantic embeddings, \textcircled{5} recognizing anatomical symmetries, \textcircled{6} generating embeddings consistent across scales, and \textcircled{7} matching anatomical structures across images of the same patient with different diseases, images of different patients, and augmented views of the same image. Adam-v2 also offers robust and generalizable representations and stands out in \textcircled{8} few-shot and \textcircled{9} full-transfer learning. These capabilities and performance directly stem from the crafted anatomy learning strategy, which explicitly constructs hierarchies for distinct anatomical structures from unlabeled medical images.

What is claimed is:

1. A method performed by a system having at least a processor and a memory therein to execute instructions for a self-supervised learning framework to learn a hierarchy of representations of human anatomy in a coarse-to-fine-manner by incorporating part-whole hierarchies into a set of learning objectives, comprising:
receiving a medical image, I, as input;
augmenting and processing, via a localizability branch of the framework, an anchor whole, w, randomly sampled from the image I, and a corresponding plurality of multi-scale views of the image I,
enforcing, via the localizability branch, a consistency between a plurality of embeddings of the plurality of multi-scale views to yield distinct features for different anatomical structures;
decomposing, via a composability branch of the framework, w into a set of parts;
enforcing, via the composability branch, a consistency between a plurality of embeddings of w and an aggregated set of embeddings of the set of parts, thereby encoding part-whole relations;
decomposing, via a decomposability branch of the framework, the plurality of embeddings of w to acquire a plurality of embeddings of its constituent parts; and
enforcing, via the decomposability branch, a consistency between the plurality of embeddings of the constituent parts and their decomposed counterparts, thereby capturing whole-part relations.

2. A framework for a self-supervised learning (SSL) model, comprising:
a localizability branch via which the model learns a semantically structured embedding space by discriminating between different anatomical structures in a medical image;
a composability branch via which the model learns part-whole relations in the anatomical structures in the medical image by constructing each anatomical structure in the medical image through an integration of constituent parts of the anatomical structure; and
a decomposability branch via which the model learns whole-part relations in the anatomical structures in the medical image by decomposing each anatomical structure in the medical image into its constituent parts.

3. The framework of claim 2, wherein the localizability branch via which the model learns the semantically structured embedding space by discriminating between different anatomical structures in the medical image, comprises the localizability branch to learn a semantically structured embedding space where similar anatomical structures are clustered together and are distinguished from dissimilar anatomical structures.

4. The framework of claim 2, wherein the localizability branch to randomly sample an anchor patch w from the medical image, extract a set C of multi-scale crops from w, and apply random data augmentations on w and the multi-scale crops in the set C to obtain an augmented view of w and augmented views of the multi-scale crops in the set C.

5. The framework of claim 4, wherein the localizability branch comprises a teacher network to receive the augmented view of w, and a student network to receive the augmented views of the multi-scale crops in the set C, to respectively generate distinct features for different anatomical structures in the medical image.

6. The framework of claim 5, wherein the localizability branch comprises a teacher head and a student head to respectively project the distinct features to output embeddings.

7. The framework of claim 2, wherein the composability branch via which the model learns part-whole relations in the anatomical structures in the medical image by constructing each anatomical structure in the medical image through the integration of constituent parts of the anatomical structure, comprises the composability branch to learn part-whole anatomical hierarchies in a bottom-up manner by assembling larger anatomical structures from their smaller constituent parts.

8. The framework of claim 2, wherein the composability branch comprises a student network and a teacher network that are shared with the localizability branch, and a composability head, wherein the composability branch to randomly sample an anchor patch w from the medical image, decompose the anchor patch w into a set of n non-overlapping parts, augment and process the parts via the student network to generate the embeddings of the parts, and concatenate and pass the embeddings to the composability head to produce aggregated embeddings of the parts.

9. The framework of claim 8, wherein the composability branch to augment and pass a whole anatomical structure in the medical image to the teacher network to generate an embedding of the whole anatomical structure, and wherein the composability branch is trained to maximize agreement between the embedding of the whole anatomical structure and the aggregated embedding of the parts.

10. The framework of claim 2, wherein the decomposability branch via which the model learns whole-part relations in the anatomical structures in the medical image by decomposing each anatomical structure in the medical image into its constituent parts, comprises the decomposability branch to learn whole-part anatomical hierarchies in a top-down manner by decomposing larger anatomical structures into smaller constituent parts.

11. The framework of claim 2, wherein the decomposability branch comprises a student network and a teacher network that are shared with the localizability branch and the composability branch, and a decomposability head,
wherein the decomposability branch to randomly sample an anchor patch w from the medical image, decompose the anchor patch w into a set of n non-overlapping parts, augment and pass the anchor patch w to the student network to generate an embedding of the whole anatomical structure, and pass the embedding of the whole anatomical structure to the decomposability head,
wherein the decomposability head to decompose the embedding of the whole anatomical structure into embeddings corresponding to the constituent parts of the whole anatomical structure, wherein the teacher network to augment and process the constituent parts to generate the embeddings of the constituent parts, and
wherein the decomposability branch is trained to maximize agreement between the embeddings of the constituent parts and the embedding of the whole anatomical structure.

12. A method involving a framework for a self-supervised learning (SSL) model, comprising:
learning, via a localizability branch of the model, a semantically structured embedding space by discriminating between different anatomical structures in a medical image;
learning, via a composability branch of the model, part-whole relations in the anatomical structures in the medical image by constructing each anatomical structure in the medical image through an integration of constituent parts of the anatomical structure; and
learning, via a decomposability branch of the model, whole-part relations in the anatomical structures in the medical image by decomposing each anatomical structure in the medical image into its constituent parts.

13. The method of claim 12, wherein learning, via the localizability branch of the model, the semantically structured embedding space by discriminating between different anatomical structures in the medical image, comprises learning, via the localizability branch, a semantically structured embedding space where similar anatomical structures are clustered together and are distinguished from dissimilar anatomical structures.

14. The method of claim 12, further comprising the localizability branch randomly sampling an anchor patch w from the medical image, extracting a set C of multi-scale crops from w, and applying random data augmentations on w and the multi-scale crops in the set C thereby obtaining an augmented view of w and augmented views of the multi-scale crops in the set C.

15. The method of claim 14, further comprising a teacher network of the localizability branch receiving the augmented view of w, and a student network of the localizability branch receiving the augmented views of the multi-scale crops in the set C, and respectively generating distinct features for different anatomical structures in the medical image.

16. The method of claim 15, wherein a teacher head and a student head of the localizability branch respectively projecting the distinct features to output embeddings.

17. The method of claim 2, wherein learning, via the composability branch of the model, part-whole relations in the anatomical structures in the medical image by constructing each anatomical structure in the medical image through the integration of constituent parts of the anatomical structure, comprises learning, via the composability branch, part-whole anatomical hierarchies in a bottom-up manner by assembling larger anatomical structures from their smaller constituent parts.

18. The method of claim 12, further comprising the composability branch randomly sampling an anchor patch w from the medical image, decomposing the anchor patch w into a set of n non-overlapping parts, augmenting and processing the parts, via a student network of the composability branch that is shared with the localizability branch, thereby generating the embeddings of the parts, and concatenating and passing the embeddings to a composability head of the localizability branch thereby producing aggregated embeddings of the parts.

19. The method of claim 18, further comprising augmenting and passing, via the composability branch, a whole anatomical structure in the medical image to a teacher network of the composability branch that is shared with the localizability branch, thereby generating an embedding of the whole anatomical structure, and training the composability branch to maximize agreement between the embedding of the whole anatomical structure and the aggregated embedding of the parts.

20. The method of claim 12, wherein learning, via the decomposability branch of the model, whole-part relations in the anatomical structures in the medical image by decomposing each anatomical structure in the medical image into its constituent parts, comprises learning, via the decomposability branch, whole-part anatomical hierarchies in a top-down manner by decomposing larger anatomical structures into smaller constituent parts.

21. The method of claim 12, further comprising:
randomly sampling, via the decomposability branch, an anchor patch w from the medical image;
decomposing the anchor patch w into a set of n non-overlapping parts;
augmenting and passing the anchor patch w to a student network of the decomposability branch that is shared with the localizability branch and the composability branch, thereby generating an embedding of the whole anatomical structure;
passing the embedding of the whole anatomical structure to a decomposability head of the decomposability branch;
decomposing the embedding of the whole anatomical structure into embeddings corresponding to the constituent parts of the whole anatomical structure;
augmenting and processing, via the teacher network, the constituent parts thereby generating the embeddings of the constituent parts; and
training the decomposability branch to maximize agreement between the embeddings of the constituent parts and the embedding of the whole anatomical structure.
